SEQUENCE LISTING (28 sequences)

SEQ ID NO: 1 - DNA, 6402 bp, Sus scrofa
  gene (1)..(6399): Factor VIII-- Full Length
  CDS (1)..(6399)
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtctcacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcggttgccaagaagcatcccaaaacctgggtgcactacatctctgcagaggaggaggactgggactacgcccccgcggtccccagccccagtgacagaagttataaaagtctctacttgaacagtggtcctcagcgaattggtaggaaatacaaaaaagctcgattcgtcgcttacacggatgtaacatttaagactcgtaaagctattccgtatgaatcaggaatcctgggacctttactttatggagaagttggagacacacttttgattatatttaagaataaagcgagccgaccatataacatctaccctcatggaatcactgatgtcagcgctttgcacccagggagacttctaaaaggttggaaacatttgaaagacatgccaattctgccaggagagactttcaagtataaatggacagtgactgtggaagatgggccaaccaagtccgatcctcggtgcctgacccgctactactcgagctccattaatctagagaaagatctggcttcgggactcattggccctctcctcatctgctacaaagaatctgtagaccaaagaggaaaccagatgatgtcagacaagagaaacgtcatcctgttttctgtattcgatgagaatcaaagctggtacctcgcagagaatattcagcgcttcctccccaatccggatggattacagccccaggatccagagttccaagcttctaacatcatgcacagcatcaatggctatgtttttgatagcttgcagctgtcggtttgtttgcacgaggtggcatactggtacattctaagtgttggagcacagacggacttcctctccgtcttcttctctggctacaccttcaaacacaaaatggtctatgaagacacactcaccctgttccccttctcaggagaaacggtcttcatgtcaatggaaaacccaggtctctgggtcctagggtgccacaactcagacttgcggaacagagggatgacagccttactgaaggtgtatagttgtgacagggacattggtgattattatgacaacacttatgaagatattccaggcttcttgctgagtggaaagaatgtcattgaacccagaagctttgcccagaattcaagaccccctagtgcgagccaaaagcaattccaaaccatcacaagtccagaagatgacgtggagcttgacccgcagtctggagagagaacccaagcactggaagaactaagtgtcccctctggtgatgggtcgatgctcttgggacagaatcctgctccacatggctcatcctcatctgatcttcaagaagccaggaatgaggctgatgattatttacctggagcaagagaaagaggcacggccccatccgcagcggcacgtctcagaccagagctgcatcacagtgccgaaagagtacttactcctgagccagagaaagagttgaagaaacttgattcaaaaatgtctagttcatcagaccttctaaagacttcgccaacaattccatcagacacgttgtcagcggagactgaaaggacacattccttaggccccccacacccgcaggttaatttcaggagtcaattaggtgccattgtacttggcaaaaattcatctcactttattggggctggtgtccctttgggctcgactgaggaggatcatgaaagctccctgggagaaaatgtatcaccagtggagagtgacgggatatttgaaaaggaaagagctcatggacctgcttcactgaccaaagacgatgttttatttaaagttaatatctctttggtaaagacaaacaaggcacgagtttacttaaaaactaatagaaagattcacattgatgacgcagctttattaactgagaatagggcatctgcaacgtttatggacaaaaatactacagcttcgggattaaatcatgtgtcaaattggataaaagggccccttggcaagaaccccctaagctcggagcgaggccccagtccagagcttctgacatcttcaggatcaggaaaatctgtgaaaggtcagagttctgggcaggggagaatacgggtggcagtggaagaggaagaactgagcaaaggcaaagagatgatgcttcccaacagcgagctcacctttctcactaactcggctgatgtccaaggaaacgatacacacagtcaaggaaaaaagtctcgggaagagatggaaaggagagaaaaattagtccaagaaaaagtcgacttgcctcaggtgtatacagcgactggaactaagaatttcctgagaaacatttttcaccaaagcactgagcccagtgtagaagggtttgatggggggtcacatgcgccggtgcctcaagacagcaggtcattaaatgattcggcagagagagcagagactcacatagcccatttctcagcaattagggaagaggcacccttggaagccccgggaaatcgaacaggtccaggtccgaggagtgcggttccccgccgcgttaagcagagcttgaaacagatcagactcccgctagaagaaataaagcctgaaaggggggtggttctgaatgccacctcaacccggtggtctgaaagcagtcctatcttacaaggagccaaaagaaataacctttctttacctttcctgaccttggaaatggccggaggtcaaggaaagatcagcgccctggggaaaagtgccgcaggcccgctggcgtccgggaagctggagaaggctgttctctcttcagcaggcttgtctgaagcatctggcaaagctgagtttcttcctaaagttcgagttcatcgggaagacctgttgcctcaaaaaaccagcaatgtttcttgcgcacacggggatctcggccaggagatcttcctgcagaaaacacggggacctgttaacctgaacaaagtaaatagacctggaaggactccctccaagcttctgggtcccccgatgcccaaagagtgggaatccctagagaagtcaccaaaaagcacagctctcaggacgaaagacatcatcagtttacccctggaccgtcacgaaagcaatcattcaatagcagcaaaaaatgaaggacaagccgagacccaaagagaagccgcctggacgaagcagggagggcctggaaggctgtgcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccacctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaaggagtgtcaggctccactgggaatggcttctggacgcattagagattttcagatcacagcttcaggacagtatggacagtgggccccaaagctggccagacttcattattccggatcaatcaatgcctggagcaccaaggatccccactcctggatcaaggtggatctgttggcaccaatgatcattcacggcatcatgacccagggtgcccgtcagaagttttccagcctctacatctcccagtttatcatcatgtacagtcttgacgggaggaactggcagagttaccgagggaattccacgggcaccttaatggtcttctttggcaatgtggacgcatctgggattaaacacaatatttttaaccctccgattgtggctcggtacatccgtttgcacccaacacattacagcatccgcagcactcttcgcatggagttgatgggctgtgatttaaacagttgcagcatgcccctgggaatgcagaataaagcgatatcagactcacagatcacggcctcctcccacctaagcaatatatttgccacctggtctccttcacaagcccgacttcacctccaggggcggacgaatgcctggcgaccccgggtgagcagcgcagaggagtggctgcaggtggacctgcagaagacggtgaaggtcacaggcatcaccacccagggcgtgaagtccctgctcagcagcatgtatgtgaaggagttcctcgtgtccagtagtcaggacggccgccgctggaccctgtttcttcaggacggccacacgaaggtttttcagggcaatcaggactcctccacccccgtggtgaacgctctggaccccccgctgttcacgcgctacctgaggatccaccccacgagctgggcgcagcacatcgccctgaggctcgaggttctaggatgtgaggcacaggatctctactga

SEQ ID NO: 2 - protein, 2133 aa, Sus scrofa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASQKQFQTITSPEDDVELDPQSGERTQALEELSVPSGDGSMLLGQNPAPHGSSSSDLQEARNEADDYLPGARERGTAPSAAARLRPELHHSAERVLTPEPEKELKKLDSKMSSSSDLLKTSPTIPSDTLSAETERTHSLGPPHPQVNFRSQLGAIVLGKNSSHFIGAGVPLGSTEEDHESSLGENVSPVESDGIFEKERAHGPASLTKDDVLFKVNISLVKTNKARVYLKTNRKIHIDDAALLTENRASATFMDKNTTASGLNHVSNWIKGPLGKNPLSSERGPSPELLTSSGSGKSVKGQSSGQGRIRVAVEEEELSKGKEMMLPNSELTFLTNSADVQGNDTHSQGKKSREEMERREKLVQEKVDLPQVYTATGTKNFLRNIFHQSTEPSVEGFDGGSHAPVPQDSRSLNDSAERAETHIAHFSAIREEAPLEAPGNRTGPGPRSAVPRRVKQSLKQIRLPLEEIKPERGVVLNATSTRWSESSPILQGAKRNNLSLPFLTLEMAGGQGKISALGKSAAGPLASGKLEKAVLSSAGLSEASGKAEFLPKVRVHREDLLPQKTSNVSCAHGDLGQEIFLQKTRGPVNLNKVNRPGRTPSKLLGPPMPKEWESLEKSPKSTALRTKDIISLPLDRHESNHSIAAKNEGQAETQREAAWTKQGGPGRLCAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 3 - DNA, 4404 bp, Sus scrofa
  gene (1)..(4401): Factor VIII-- B-domain deleted
  CDS (1)..(4401)
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcggttgccaagaagcatcccaaaacctgggtgcactacatctctgcagaggaggaggactgggactacgcccccgcggtccccagccccagtgacagaagttataaaagtctctacttgaacagtggtcctcagcgaattggtaggaaatacaaaaaagctcgattcgtcgcttacacggatgtaacatttaagactcgtaaagctattccgtatgaatcaggaatcctgggacctttactttatggagaagttggagacacacttttgattatatttaagaataaagcgagccgaccatataacatctaccctcatggaatcactgatgtcagcgctttgcacccagggagacttctaaaaggttggaaacatttgaaagacatgccaattctgccaggagagactttcaagtataaatggacagtgactgtggaagatgggccaaccaagtccgatcctcggtgcctgacccgctactactcgagctccattaatctagagaaagatctggcttcgggactcattggccctctcctcatctgctacaaagaatctgtagaccaaagaggaaaccagatgatgtcagacaagagaaacgtcatcctgttttctgtattcgatgagaatcaaagctggtacctcgcagagaatattcagcgcttcctccccaatccggatggattacagccccaggatccagagttccaagcttctaacatcatgcacagcatcaatggctatgtttttgatagcttgcagctgtcggtttgtttgcacgaggtggcatactggtacattctaagtgttggagcacagacggacttcctctccgtcttcttctctggctacaccttcaaacacaaaatggtctatgaagacacactcaccctgttccccttctcaggagaaacggtcttcatgtcaatggaaaacccaggtctctgggtccttgggtgccacaactcagacttgcggaacagagggatgacagccttactgaaggtgtatagttgtgacagggacattggtgattattatgacaacacttatgaagatattccaggcttcttgctgagtggaaagaatgtcattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaaggagtgtcaggctccactgggaatggcttctggacgcattagagattttcagatcacagcttcaggacagtatggacagtgggccccaaagctggccagacttcattattccggatcaatcaatgcctggagcaccaaggatccccactcctggatcaaggtggatctgttggcaccaatgatcattcacggcatcatgacccagggtgcccgtcagaagttttccagcctctacatctcccagtttatcatcatgtacagtcttgacgggaggaactggcagagttaccgagggaattccacgggcaccttaatggtcttctttggcaatgtggacgcatctgggattaaacacaatatttttaaccctccgattgtggctcggtacatccgtttgcacccaacacattacagcatccgcagcactcttcgcatggagttgatgggctgtgatttaaacagttgcagcatgcccctgggaatgcagaataaagcgatatcagactcacagatcacggcctcctcccacctaagcaatatatttgccacctggtctccttcacaagcccgacttcacctccaggggcggacgaatgcctggcgaccccgggtgagcagcgcagaggagtggctgcaggtggacctgcagaagacggtgaaggtcacaggcatcaccacccagggcgtgaagtccctgctcagcagcatgtatgtgaaggagttcctcgtgtccagtagtcaggacggccgccgctggaccctgtttcttcaggacggccacacgaaggtttttcagggcaatcaggactcctccacccccgtggtgaacgctctggaccccccgctgttcacgcgctacctgaggatccaccccacgagctgggcgcagcacatcgccctgaggctcgaggttctaggatgtgaggcacaggatctctactga

SEQ ID NO: 4 - protein, 1467 aa, Sus scrofa
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 5 - DNA, 9009 bp, Homo sapiens
  misc_feature: Factor VIII- Full Length
  CDS (208)..(7203)
cagtgggtaagttccttaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccattggtcttaattctgaccaataaaaaaataagtcaggaggatgcaattgttgaaagctttgaaataaaataacatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgcaccccttcactatgactacacagaactctcctgatagtaaagggggctggaggcaaggataagttatagagcagttggaggaagcatccaaagactgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacataca

SEQ ID NO: 6 - protein, 2332 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - DNA, 6996 bp, Homo sapiens
  misc_feature: Factor VIII- Full Length coding sequence
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 8 - protein, 2319 aa, Mus musculus
MQIALFACFFLSLFNFCSSAIRRYYLGAVELSWNYIQSDLLSVLHTDSRFLPRMSTSFPFNTSIMYKKTVFVEYKDQLFNIAKPRPPWMGLLGPTIWTEVHDTVVITLKNMASHPVSLHAVGVSYWKASEGDEYEDQTSQMEKEDDKVFPGESHTYVWQVLKENGPMASDPPCLTYSYMSHVDLVKDLNSGLIGALLVCKEGSLSKERTQMLYQFVLLFAVFDEGKSWHSETNDSYTQSMDSASARDWPKMHTVNGYVNRSLPGLIGCHRKSVYWHVIGMGTTPEIHSIFLEGHTFFVRNHRQASLEISPITFLTAQTLLIDLGQFLLFCHISSHKHDGMEAYVKVDSCPEESQWQKKNNNEEMEDYDDDLYSEMDMFTLDYDSSPFIQIRSVAKKYPKTWIHYISAEEEDWDYAPSVPTSDNGSYKSQYLSNGPHRIGRKYKKVRFIAYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIYEDIPTQLVNENNVIDPRSFFQNTNHPNTRKKKFKDSTIPKNDMEKIEPQFEEIAEMLKVQSVSVSDMLMLLGQSHPTPHGLFLSDGQEAIYEAIHDDHSPNAIDSNEGPSKVTQLRPESHHSEKIVFTPQPGLQLRSNKSLETTIEVKWKKLGLQVSSLPSNLMTTTILSDNLKATFEKTDSSGFPDMPVHSSSKLSTTAFGKKAYSLVGSHVPLNASEENSDSNILDSTLMYSQESLPRDNILSIENDRLLREKRFHGIALLTKDNTLFKDNVSLMKTNKTYNHSTTNEKLHTESPTSIENSTTDLQDAILKVNSEIQEVTALIHDGTLLGKNSTYLRLNHMLNRTTSTKNKDIFHRKDEDPIPQDEENTIMPFSKMLFLSESSNWFKKTNGNNSLNSEQEHSPKQLVYLMFKKYVKNQSFLSEKNKVTVEQDGFTKNIGLKDMAFPHNMSIFLTTLSNVHENGRHNQEKNIQEEIEKEALIEEKVVLPQVHEATGSKNFLKDILILGTRQNISLYEVHVPVLQNITSINNSTNTVQIHMEHFFKRRKDKETNSEGLVNKTREMVKNYPSQKNITTQRSKRALGQFRLSTQWLKTINCSTQCIIKQIDHSKEMKKFITKSSLSDSSVIKSTTQTNSSDSHIVKTSAFPPIDLKRSPFQNKFSHVQASSYIYDFKTKSSRIQESNNFLKETKINNPSLAILPWNMFIDQGKFTSPGKSNTNSVTYKKRENIIFLKPTLPEESGKIELLPQVSIQEEEILPTETSHGSPGHLNLMKEVFLQKIQGPTKWNKAKRHGESIKGKTESSKNTRSKLLNHHAWDYHYAAQIPKDMWKSKEKSPEIISIKQEDTILSLRPHGNSHSIGANEKQNWPQRETTWVKQGQTQRTCSQIPPVLKRHQRELSAFQSEQEATDYDDAITIETIEDFDIYSEDIKQGPRSFQQKTRHYFIAAVERLWDYGMSTSHVLRNRYQSDNVPQFKKVVFQEFTDGSFSQPLYRGELNEHLGLLGPYIRAEVEDNIMVTFKNQASRPYSFYSSLISYKEDQRGEEPRRNFVKPNETKIYFWKVQHHMAPTEDEFDCKAWAYFSDVDLERDMHSGLIGPLLICHANTLNPAHGRQVSVQEFALLFTIFDETKSWYFTENVKRNCKTPCNFQMEDPTLKENYRFHAINGYVMDTLPGLVMAQDQRIRWYLLSMGNNENIQSIHFSGHVFTVRKKEEYKMAVYNLYPGVFETLEMIPSRAGIWRVECLIGEHLQAGMSTLFLVYSKQCQIPLGMASGSIRDFQITASGHYGQWAPNLARLHYSGSINAWSTKEPFSWIKVDLLAPMIVHGIKTQGARQKFSSLYISQFIIMYSLDGKKWLSYQGNSTGTLMVFFGNVDSSGIKHNSFNPPIIARYIRLHPTHSSIRSTLRMELMGCDLNSCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 9 - protein, 14 aa, Homo sapeins
  DOMAIN (1)..(14): Linker sequence
SFSQNPPVLKRHQR

SEQ ID NO: 10 - protein, 4 aa, Homo sapiens
RHQR

SEQ ID NO: 11 - protein, 24 aa, Sus Scrofa
  DOMAIN (1)..(24): Linker sequence
SFAQNSRPPSASAPKPPVLRRHQR

SEQ ID NO: 12 - DNA, 4371 bp, Homo sapiens
  CDS (1)..(4371): B domain-deleted factor VIII (HSQ)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagcttctctcagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 13 - protein, 1457 aa, Homo sapiens
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 14 - protein, 1467 aa, Artificial Sequence
  Synthetic ET-3 Factor VIII
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 15 - protein, 1467 aa, Artificial Sequence
  Synthetic ETX-A1-fV
MQLELSTCVFLCLLPLGFSATRRYYLGAVELSWDYRQSDLGELPVDARFPPRVPKSFPFNTSVVYKKTVFVEFTDHLFNIAKPRPPWMGLLGPTIQAEVYDTVVVTLKNMASHPVSLHAVGVSYWKASEGAEYDDHTSQREKEDDKVFPGGSHTYVWQVLKENGPTASDPPCLTYSYLSHVDLVKDLNSGLIGALLVCREGSLTKEKTQTLHKFVLLFAVFDEGKSWHSETKNSLMQDRDAASARAWPKMHTVNGYVNRSLPGLIGCHRKSVYWHVIGMGTTPEVHSIFLEGHTFLVRHHRQASLEISPITFLTAQTLLMDLGQFLLFCHISSHQHDGMEAYVKVDSCPEEPQLRMKANEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - protein, 1467 aa, Artificial Sequence
  Synthetic ETX-A3-Cu
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17 - protein, 1467 aa, Artificial Sequence
  Synthetic ETX-A3-fV
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKKTRHYFIAAVERLWDYGMSESPHVLRNRAQSGSVPQFKKVVFREFTDGSFTQPLYRGELNEHLGLLGPYIRAEVEDNIMVTFKNQASRPYSFYSSLISYEEDQRQGAEPRKNFVQPNETKTYFWKVQHHMAPTKDEFDCKAWAYFSDVDLEKDVHSGLIGPLLICHTNTLNPAHGRQVTVQEFALFFTIFDETKSWYFTENMERNCRAPCNLQMEDPTFKENYRFHAINGYIMDTLPGLVMAQDQRIRWYLLSMGSNENIHSIHFSGHVFTVRKKEEYKMAVYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - protein, 1467 aa, Artificial Sequence
  Synthetic ETX-A3-all
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKKTRHYFIAAVERLWDYGMSESPHVLRNRAQNGEVPQFKKVVFREFTDGSFTQPLYRGELNEHLGLLGPYIRAEVEDNIMVTFKNQASRPYSFYSSLISYEEDQRQGAEPRKNFVQPNETKTYFWKVQHHMAPTKDEFDCKAWAYFSDVDLEKDVHSGLIGPLLICRTNTLNPAHGRQVTVQEFALFFTIFDETKSWYFTENMERNCRAPCNLQMEDPTFKENYRFHAINGYIMDTLPGLVMAQDQRIRWYLLSMGSNENIHSIHFSGHVFTVRKKEEYKMAVYNLYPGVFETVEMLPSKAGIWRVECLIGEHLHAGMSTTFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 1467 aa, Artificial Sequence
  Synthetic ETX-hSP
MQIELSTCFFLCLLRFCFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 20 - protein, 1457 aa, Artificial Sequence
  Synthetic ETX-SQ
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 21 - protein, 1457 aa, Artificial Sequence
  Synthetic ETX-hSP-SQ
MQIELSTCFFLCLLRFCFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 22 - protein, 371 aa, Artificial Sequence
  Synthetic ETx s7-8 (A1 domain of HSQ)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPLTFLTAQTFLMDLGQFLLFCHISSHHHGGMEAHVRVESCAEEPQLRRKADEEEDYDDNLYDSDMDVVRLDGDDVSPFIQIR

SEQ ID NO: 23 - protein, 372 aa, Artificial Sequence
  Synthetic ETX A1-fV (A1 domain of HSQ)
ATRRYYLGAVELSWDYRQSDLGELPVDARFPPRVPKSFPFNTSVVYKKTVFVEFTDHLFNIAKPRPPWMGLLGPTIQAEVYDTVVVTLKNMASHPVSLHAVGVSYWKASEGAEYDDHTSQREKEDDKVFPGGSHTYVWQVLKENGPTASDPPCLTYSYLSHVDLVKDLNSGLIGALLVCREGSLTKEKTQTLHKFVLLFAVFDEGKSWHSETKNSLMQDRDAASARAWPKMHTVNGYVNRSLPGLIGCHRKSVYWHVIGMGTTPEVHSIFLEGHTFLVRHHRQASLEISPITFLTAQTLLMDLGQFLLFCHISSHQHDGMEAYVKVDSCPEEPQLRMKANEEAEDYDDDLTDSEMDVVRFDDDNSPSFIQIR

SEQ ID NO: 24 - protein, 372 aa, Artificial Sequence
  Synthetic ETX A1 All (A1 domain of HSQ)
ATRRYYLGAVELSWDYRQSDLGELPVDTRFPPRVPKSFPFNTSVLYKKTVFVEFTDHLFNIAKPRPPWMGLLGPTIQAEVYDTVVVTLKNMASHPVSLHAVGVSYWKASEGAEYDDHTSQREKEDDKVFPGGSHTYVWQVLKENGPTASDPPCLTYSYLSHVDLVKDLNSGLIGALLVCREGSLTKEKTQTLHEFVLLFAVFDEGKSWHSETKDSLMQDRDAASARAWPKMHTVNGYVNRSLPGLIGCHRKSVYWHVIGMGTTPEVHSIFLEGHTFLVRHHRQASLEISPITFLTAQTLLMDLGQFLLFCHISSHQHDGMEAYVKVDSCPEEPQLRMKANEEAEDYDDNLTDSEMDVVRFDDDNSPSFIQIR

SEQ ID NO: 25 - protein, 330 aa, Artificial Sequence
  Synthetic ETX A3 fV (A3 domain of HSQ)
SFQKKTRHYFIAAVERLWDYGMSESPHVLRNRAQSGSVPQFKKVVFREFTDGSFTQPLYRGELNEHLGLLGPYIRAEVEDNIMVTFKNQASRPYSFYSSLISYEEDQRQGAEPRKNFVQPNETKTYFWKVQHHMAPTKDEFDCKAWAYFSDVDLEKDVHSGLIGPLLICHTNTLNPAHGRQVTVQEFALFFTIFDETKSWYFTENMERNCRAPCNLQMEDPTFKENYRFHAINGYIMDTLPGLVMAQDQRIRWYLLSMGSNENIHSIHFSGHVFTVRKKEEYKMAVYNLYPGVFETVEMLPSKAGIWRVECLIGEHLHAGMSTLFLVYSN

SEQ ID NO: 26 - protein, 330 aa, Artificial Sequence
  Synthetic ETX A3 all (A3 domain of HSQ)
SFQKKTRHYFIAAVERLWDYGMSESPHVLRNRAQNGEVPQFKKVVFREFTDGSFTQPLYRGELNEHLGLLGPYIRAEVEDNIMVTFKNQASRPYSFYSSLISYEEDQRQGAEPRKNFVQPNETKTYFWKVQHHMAPTKDEFDCKAWAYFSDVDLEKDVHSGLIGPLLICRTNTLNPAHGRQVTVQEFALFFTIFDETKSWYFTENMERNCRAPCNLQMEDPTFKENYRFHAINGYIMDTLPGLVMAQDQRIRWYLLSMGSNENIHSIHFSGHVFTVRKKEEYKMAVYNLYPGVFETVEMLPSKAGIWRVECLIGEHLHAGMSTTFLVYSN

SEQ ID NO: 27 - protein, 330 aa, Artificial Sequence
  Synthetic ETX A3 Cu (A3 domain of HSQ)
SFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFSVRKKEEYKMAVYNLYPGVFETVEMLPSKVGIWRIECLIGEHLQAGMSTTFLVYSK

SEQ ID NO: 28 - protein, 1457 aa, Artificial Sequence
  Synthetic HSQ fVIII
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2023/0357360 A1
(43) Pub. Date: Nov. 9, 2023

(54) NUCLEIC ACID AND AMINO ACID SEQUENCES ENCODING HIGH-LEVEL EXPRESSOR FACTOR VIII POLYPEPTIDES AND METHODS OF USE

(71) Applicant: Expression Therapeutics, LLC, Tucker, GA (US)

(72) Inventor: Harrison Brown, Atlanta, GA (US)

(21) Appl. No.: 17/632,222

(22) PCT Filed: Jan. 31, 2022
(86) PCT No.: PCT/US22/14639

§ 371 Date: Feb. 1, 2022

Related U.S. Application Data
(60) Provisional application No. 63/143,315, filed on Jan. 29, 2021.

(51) Int. Cl. C07K 14/755; A61P 7/04

(52) U.S. Cl. CPC C07K 14/755; A61P 7/04; A61K 38/00

(57) ABSTRACT

Methods and compositions are provided that allow for high-level expression of a factor VIII polypeptide. More specifically, methods and compositions are provided comprising nucleic acid and amino acid sequences comprising a modified factor VIII that result in high-level expression of the polypeptide. The methods and compositions of the invention find use in the treatment of factor VIII deficiency including, for example, hemophilia A.

### CROSS REFERENCE TO RELATED APPLICATIONS

This application is a National Stage of International Application No. PCT/US22/14639, International Filing Date of Jan. 31, 2022, which claims priority to U.S. Provisional Application No. 63/143,315 filed Jan. 29, 2021, incorporated by reference in its entirety.

### SEQUENCE LISTING

The nucleic acid and amino acid sequences accompanying the sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “103_390USSequence.txt” (˜342 kb), which was created on Jan. 28, 2022, which is incorporated by reference herein.

### BACKGROUND

Factor VIII is a large (˜300 kDa) glycoprotein that functions as an integral component of the intrinsic pathway of blood coagulation. It contains a series of domains designated A1-A2-B-ap-A3-C1-C2. The B domain of factor VIII has no known function and can be deleted without loss of coagulant activity. Mutations in the factor VIII gene that result in decreased or defective factor VIII protein give rise to the genetic disease, hemophilia A, which is characterized by recurrent bleeding episodes. Treatment of hemophilia A requires intravenous infusion of either plasma-derived or recombinant factor VIII.

Since the introduction of recombinant factor VIII for the treatment of hemophilia A, supply has struggled to keep up with demand because factor VIII is expressed and recovered at low levels in the heterologous mammalian cell culture systems used for commercial manufacture (Garber et al. (2000) Nature Biotechnology 18: 1133). Additionally, factor VIII levels during hemophilia A gene therapy trials indicate that expression levels will be a limiting feature (Roth, et al. (2001) N. Engl. J. Med. 344:1735-1742). The importance of this problem has resulted in significant research efforts to overcome the low factor VIII expression barrier. Several factors that limit expression have been identified, including low mRNA levels (Lynch et al. (1993) Hum. Gene Ther. 4:259-272; Hoeben et al. (1995) Blood 85:2447-2454; Koeberl et al. (1995) Hum. Gene Ther. 6:469-479), interaction with protein chaperones and inefficient secretion (Pipe et al. (1998) J. Biol. Chem. 273:8537-8544; Tagliavacca et al. (2000) Biochemistry 39:1973-1981; Kaufman et al. (1997) Blood Coagul Fibrinolysis 8 Suppl 2:S3-14) and rapid decay in the absence of von Willebrand factor (Kaufman et al. (1988) J. Biol. Chem. 263:6352-6362 and Kaufman et al. (1989) Mol. Cell Biol. 9:1233-1242). Deletion of the B-domain has been shown to increase factor VIII protein production in heterologous systems (Toole et al. (1986) Proc. Natl. Acad. Sci. U.S.A. 83:5939-5942). A B-domain deleted form of human factor VIII (Lind et al. (1995) Eur. J. Biochem. 232:19-27) has been approved for clinical use.

Despite these insights into factor VIII regulation, expression continues to be significantly lower than other recombinant proteins in the heterologous systems used in commercial manufacture (Kaufman et al. (1997) Blood Coagul. Fibrinolysis 8 Suppl 2:S3-14), as well as in ex-vivo (Roth, et al. (2001) N Engl. J. Med. 344:1735-1742) and in vivo gene therapy applications (Chuah et al. (1995) Hum. Gene Ther. 6:1363-1377). Methods and compositions are needed for the increased expression of factor VIII.

### BRIEF DESCRIPTION OF THE FIGURES

FIGS. 1A and 1B illustrate a sequence alignment of the A1 and A3 domains for human and porcine orthologues of the ET3 variant fVIII protein. (FIG. 1A) Sequence alignment of the A1 domain for human (upper sequence, SEQ ID NO: 6) and porcine (middle sequence, SEQ ID NO: 2) ET3 variant of fVIII (bottom sequence, SEQ ID NO: 14). The lower sequence shows identical residues. Amino acid sequence alignments for the signal peptide (N-terminal bar), heavy chain acidic domain (C-terminal bar), human (top) and ET3 (bottom) fVIII are shown. Disulfide linkages are noted by the lines connecting cysteine residues. Places where either human, ET3 or both sequences encode an N-linked glycosylation attachment site (N-X-S/T) are outlined with a box. (FIG. 1B) Sequence alignment of the A3 domain for human (upper sequence, SEQ ID NO: 6) and porcine (middle sequence, SEQ ID NO: 2) ET3 variant of fVIII (bottom sequence, SEQ ID NO: 14). The lower sequence shows identical residues. Amino acid sequence alignments for the activation peptide (bar), human (top) and ET3 (bottom) fVIII are shown. Disulfide linkages are noted by the lines connecting cysteine residues. Places where either human, ET3 or both sequences encode an N-linked glycosylation attachment site (N-X-S/T) are outlined with a box.

FIG. 2A shows in vitro expression data in HepG2 cells indicating that liver specific codon optimization improves expression for the HSQ and ET3 variants of the fVIII protein.

FIG. 2B shows in vivo data for codon optimized HSQ and ET3 indicating increased expression of fVIII following hydrodynamic injection of an AAV-vector encoding liver codon optimized variants of the HSQ and ET3 fVIII protein into mice.

FIG. 3 shows in vitro expression data in HepG2 cells indicating retained expression above HSQ (SEQ ID NO: 28) for the ETX-A1-FV (SEQ ID NO: 15), ETX-A3-Cu (SEQ ID NO: 16), ETX-A3-FV (SEQ ID NO: 17), ETX-A3-A3-All (SEQ ID NO: 18), ETX-hSP (SEQ ID NO: 19) and ETX-SQ (SEQ ID NO: 20) variants and retained expression at ET3 levels with further humanization for the ETX-hSP (SEQ ID NO: 19) and ETX-SQ (SEQ ID NO: 20) variants of the fVIII protein.

FIG. 4 illustrates domain maps of the fVIII variants HSQ (SEQ ID NO: 28); ET3 (SEQ ID NO: 14); ETX-hSP (SEQ ID NO: 19); and ETX-hSP-SQ (SEQ ID NO: 21).

### SUMMARY

Methods and compositions are provided that allow for high-level expression of a factor VIII polypeptide. More specifically, we disclose methods and compositions comprising nucleic acid and amino acid sequences comprising a modified factor VIII that results in high-level expression of the polypeptide. The methods and compositions find use in the treatment of factor VIII deficiency, including, for example, hemophilia A.

In particular, one variation provides an isolated polypeptide comprising an amino acid sequence set forth in the sequences ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), AND ETX-hSP-SQ (SEQ ID NO: 21); an amino acid sequence having at least 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), AND ETX-hSP-SQ (SEQ ID NO: 21), wherein said polypeptide is characterized by high-level expression, or a fragment thereof.

In another variation, we disclose ETX-hSP (SEQ ID NO: 19) and ETX-SQ (SEQ ID NO: 20). ETX-hSP (SEQ ID NO: 19) and ETX-SQ (SEQ ID NO: 20) are two novel variants of the ET3 molecule, which are more humanized that ET3. “hSP” refers to “human signal peptide”. The ET3 molecule uses the porcine signal peptide. ETX-hSP (SEQ ID NO: 19) replaces this porcine signal peptide in ET3 with the human signal peptide. Our data show no difference in FVIII expression when the substitution is made, indicating that we can maintain increased expression while making a more human FVIII construct.

“SQ” in ETX-SQ (SEQ ID NO: 20) refers to the linker region used to replace the FVIII B-domain in B-domain deleted FVIII. The SQ linker sequence is derived human B-domain sequence to retain key glycosylation sites found in the B-domain. ET3 uses a similar, porcine-derived linker sequence designated “OL”. OL also retains key glycosylation sites found in the porcine B-domain and is derived from the porcine B-domain sequence. Importantly, the OL linker sequence is 30 base pairs longer than the SQ linker. This makes substituting SQ into ET3 an attractive approach for AAV therapies, where the limited cargo capacity of AAV favors shorter transgene designs.

We finally disclose an ETX-hSP-SQ (SEQ ID NO: 21) molecule that combines the two substitutions. The hybrid molecule retains the same levels FVIII expression seen in ET3 while further humanizing the construct. The dual ETx-hSP-SQ would have 91% sequence identity to the B-domain deleted human FVIII HSQ.

In another embodiment of the invention, isolated nucleic acid molecules are provided comprising a nucleotide sequence encoding a polypeptide comprising the amino acid sequence set forth in ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), AND ETX-hSP-SQ (SEQ ID NO: 21); and, a nucleotide sequence having at least 85%, 90%, 95%, 97%, 98%, or 99% sequence identity to the nucleotide sequence encoding a polypeptide 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), wherein said nucleotide sequence encodes a polypeptide that is characterized by high-level expression. Expression cassettes, vectors, and cells comprising the nucleic acid molecules of the invention are further provided.

Pharmaceutical compositions comprising the nucleic acid molecules and the polypeptides of the invention are also provided.

Methods for the production of a polypeptide are provided. In one embodiment, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), wherein the nucleotide sequence encodes a polypeptide characterized by high-level expression, or a fragment thereof; and, culturing the cell under conditions that allow expression of the nucleotide sequence.

Also provided are methods for increasing the level of expression of the factor VIII polypeptide. In one variation, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21); wherein the nucleotide sequence encodes a polypeptide characterized by high-level expression, or a fragment thereof; and, culturing the cell under conditions that allow expression of the nucleotide sequence.

Also provided is a method for the treatment of factor VIII deficiencies, including, for example, hemophilia A. The method comprises administering to a subject in need thereof a composition comprising a therapeutically effective amount of a polypeptide, where the polypeptide comprises an amino acid sequence set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), an amino acid sequence having at least 85%, 90%, 95%, 97%, 98%, or 99% sequence identity to 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), wherein said polypeptide is characterized by high-level expression, or a fragment thereof.

Other methods include treating a factor VIII deficiency by administering to a subject in need thereof a composition comprising a therapeutically effective amount of a nucleic acid molecule, where said nucleic acid molecule comprises a nucleotide sequence set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21); a nucleotide sequence encoding a polypeptide comprising the amino acid sequence set forth in ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21); a nucleotide sequence having at least 85%, 90%, 95%, 97%, 98%, or 99% sequence identity to 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), wherein said nucleic acid molecule encodes a polypeptide characterized by high-level expression, or a fragment thereof.

### DETAILED DESCRIPTION

### Overview

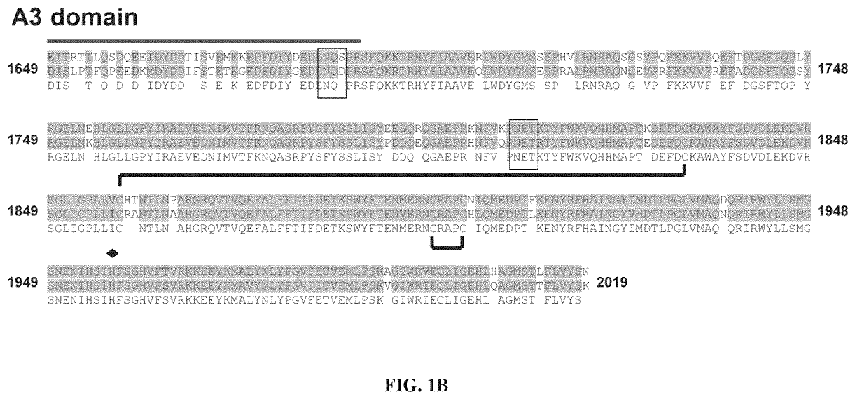

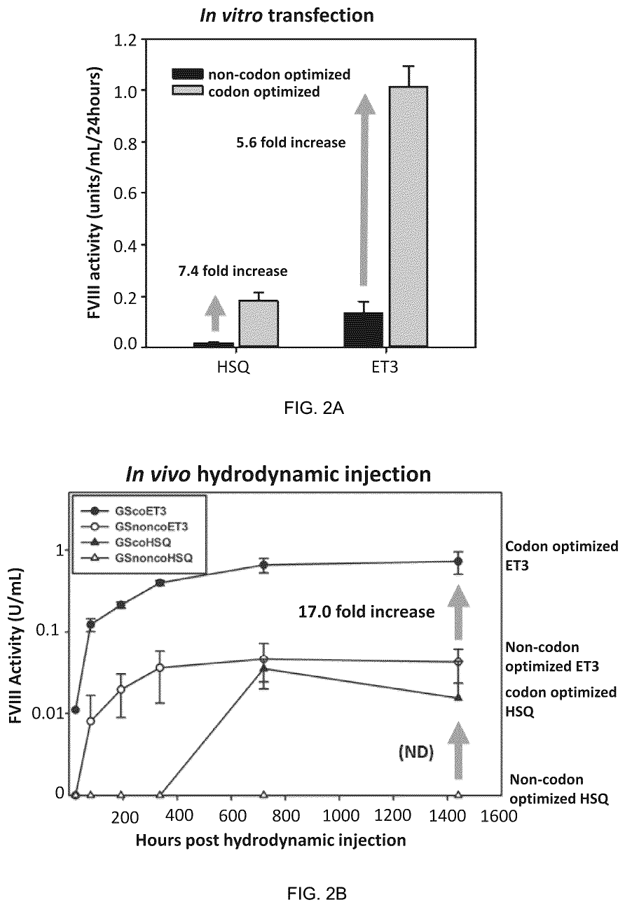

We provide methods and compositions that allow for high-level expression of the factor VIII polypeptide. We further provide more humanized versions of factor VIII, which retain the high levels of expression achieved by the factor VIII variants HSQ (SEQ ID NO: 28) and ET3 (SEQ ID NO: 14). (See FIGS. 2A and 2B to see comparison of HSQ and ET3 with human FVIII). The factor VIII polypeptide contains homology-defined proteins domains having the following nomenclature: A1-A2-B-ap-A3-C1-C2. We have identified regions within the domains of a non-human factor VIII polypeptide that promote high-level expression of the factor VIII polypeptide. More particularly, regions of the porcine factor VIII polypeptide that comprises the A1 and ap-A3 regions, and variants and fragments thereof, have been identified which impart high-level expression to both the porcine and human factor VIII polypeptide. We thus provide methods and compositions that use the non-human factor VIII polypeptide sequences which impart high-level expression, and active variants or fragments of these sequences, to construct novel nucleic acid and polypeptide sequences encoding a modified factor VIII polypeptide that results in high-level expression of the encoded factor VIII polypeptide. The modified factor VIII polypeptides characterized by high-level expression are referred to herein as “factor VIII_{SEP}” (Super Expression).

By “high-level expression” is intended the production of a polypeptide at increased levels when compared to the expression levels of the corresponding human factor VIII polypeptide (See, e.g., FIGS. 2A and 2B which show comparison of HSQ, SEQ ID NO: 28 versus human fVIII) expressed under the same conditions. An increase in polypeptide levels (i.e., high-level expression) comprises at least about 0.5, 1, 2, 3, 4, 5, 6, 7, 8, 9, 10, 11, 12, 13, 14, 15, 20 fold or greater expression of the factor VIII_{SEP} polypeptide compared to the expression levels of the corresponding human factor VIII polypeptide. See FIGS. 2A and 2B to see comparison of HSQ and human FVIII. Alternatively, “high-level expression” can comprise an increase in polypeptide expression levels of at least 1-25 fold, 1-5 fold, 5-10 fold, 10-15 fold, 15-20 fold, 20-25 fold or greater expression levels of the factor VIII_{SEP} when compared to the corresponding human factor VIII polypeptide (See, e.g., FIGS. 2A and 2B which show comparison of HSQ, SEQ ID NO: 28 versus human fVIII). Methods for assaying “high-level expression” are routine in the art and are outlined in more detail below.

By “corresponding” factor VIII polypeptide is intended a factor VIII polypeptide that comprises an equivalent amino acid sequence. For instance, when a modified factor VIII polypeptide comprising the A1-A2-ap-A3-C1-C2 domains is tested for high-level expression, a human or porcine factor VIII polypeptide containing corresponding domains will be used (i.e., A1-A2-ap-A3-C1-C2). When a fragment of a modified factor VIII polypeptide is tested for high-level expression (i.e., A1-A2-ap-A3), a human or porcine factor VIII polypeptide having the corresponding domains will be tested (i.e., A1-A2-ap-A3).

### Compositions

Compositions of the invention include the nucleic acid molecules encoding factor VIII polypeptides characterized by high-level expression. As outlined in further detail below, the A1 domain of porcine factor VIII (amino acid residues 20-391 of SEQ ID NO:2) and the ap-A3 domain of porcine factor VIII (amino acids 1450-1820 of SEQ ID NO:2) allow for high-level expression of factor VIII. The present invention thus provides methods and compositions comprising factor VIII_{SEP} polypeptides and active variant and active fragments of factor VIII_{SEP} polypeptides characterized by high-level expression.

In particular, the present invention provides for isolated nucleic acid molecules comprising nucleotide sequences encoding the amino acid sequence shown in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) and active fragments or active variants thereof. Also provided are isolated nucleic acid molecules comprising nucleotide sequences that code for 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) and active fragments or active variants thereof. Further provided are polypeptides having an amino acid sequence encoded by a nucleic acid molecule described herein, for example, those set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) and active fragments and active variants thereof.

We disclose isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed factor VIII_{SEP} nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the polypeptides set forth in 85%, 90%, 95%, 97%, 98%, or 99% sequence identity to ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) and hence are characterized by high-level expression of the factor VIII polypeptide. Thus, fragments of a nucleotide sequence may range from at least about 10, about 20 nucleotides, about 50 nucleotides, about 100 nucleotides, about 500 nucleotides, about 1000 nucleotides, about 2000 nucleotides, about 3000 nucleotides, about 4000 nucleotides, about 5000 nucleotides, about 6000 nucleotides, about 7000 nucleotides, about 8000 nucleotides, and up to the full-length nucleotide sequence encoding the factor VIII polypeptide of the invention about 9000 nucleotides.

A fragment of a nucleotide sequence of the present invention that encodes a biologically active portion of a factor VIII_{SEP} protein of the invention will encode at least 12, 25, 30, 50, 100, 150, 200, 300, 400, 500, 600, 700, 800, 900, 1000, 1100, 1200, 1300, 1400, 1500, 1600, 1700, 1800, 1900, 2000, 2100, 2200, 2300 contiguous amino acids, or up to the total number of amino acids present in a full-length factor VIII protein of the invention (for example, approximately 1400 to approximately 1600 amino acids for ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) and will allow high-level expression of the factor VIII polypeptide.

By “variant” is intended substantially similar sequences. For nucleotide sequences, conservative variants include those sequences that, because of the degeneracy of the genetic code, encode the amino acid sequence of one of the polypeptides of the invention. Variant nucleotide sequences include synthetically derived nucleotide sequences, such as those generated, for example, by using site-directed mutagenesis but which still encode a factor VIII_{SEP} protein characterized by high-level expression. Generally, variants of a particular nucleotide sequence of the invention will have at least at least 75%, 80%, 85%, 86%, 87%, 88%, 89%, preferably about 90%, 91%, 92%, 93%, 94%, 95%, 96%, 97%, and more preferably about 98%, 99%, or more sequence identity to that particular nucleotide sequence as determined by sequence alignment programs described elsewhere herein.

By “variant” protein is intended a protein derived from the polypeptide of ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) by deletion (so-called truncation) or addition of one or more amino acids to the N-terminal and/or C-terminal end of the protein; deletion or addition of one or more amino acids at one or more sites in the protein; or substitution of one or more amino acids at one or more sites in the protein. Variant proteins encompassed by the present invention are biologically active, that is they continue to possess the desired biological activity of ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21), hence they will continue to allow for the high-level expression of the factor VIII polypeptide. Such variants may result from, for example, genetic polymorphism or from human manipulation. Biologically active variants of a polypeptide of the invention will have at least 75%, 80%, 85%, 86%, 87%, 88%, 89%, preferably about 90%, 91%, 92%, 93%, 94%, 95%, 96%, 97%, 98%, 99% or more sequence identity to the amino acid sequence for ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) as determined by sequence alignment programs described elsewhere herein using default parameters. A biologically active variant of a protein of the invention may differ from that protein by as few as 1-100, 1-50, 1-25, 1-15 amino acid residues, as few as 1-10, such as 6-10, as few as 5, as few as 4, 3, 2, or even 1 amino acid residue.

Biological activity of the factor VIII_{SEP} polypeptides can be assayed by any method known in the art. As discussed above, the factor VIII_{SEP} polypeptides are characterized by high-level expression. Assays to measure high-level expression are known in the art. For example, the level of expression of the factor VIII_{SEP} polypeptide can be measured by Western blot analysis or ELISA. Other methods include, for example, labeling cell lines expressing the factor VIII polypeptide with ³⁵S-ethionine, followed by immunoprecipitation of radiolabeled factor VIII molecules. Alternatively, the level of expression of the factor VIII_{SEP} polypeptide can be assayed for by measuring the activity of the factor VIII polypeptide. For example, increased factor VIII expression could be assayed by measuring factor VIII activity using standard assays known in the art, including a one-stage coagulation assay or a two-stage activity assay. See, for example, U.S. Pat. No. 6,458,561 and the Experimental section below.

Briefly, coagulation assays are based on the ability of factor VIII to shorten the clotting time of plasma derived from a patient with hemophilia A. For example, in the one-stage assay, 0.1 ml hemophilia A plasma (George King Biomedical, Inc.) is incubated with 0.1 ml activated partial thromboplastin reagent (APTT) (Organon Teknika) and 0.01 ml sample or standard, consisting of diluted, citrated normal human plasma, for 5 min at 37° C. in a water bath. Incubation is followed by addition of 0.1 ml 20 mM CaCl₂), and the time for development of a fibrin clot is determined by visual inspection. A unit of factor VIII is defined as the amount present in 1 ml of citrated normal human plasma.

The one-stage assay relies on endogenous activation of factor VIII by activators formed in the hemophilia A plasma, whereas the two-stage assay measures the procoagulant activity of preactivated factor VIII. In the two-stage assay, samples containing factor VIII that are reacted with thrombin are added to a mixture of activated partial thromboplastin and human hemophilia A plasma that is preincubated for 5 min at 37° C. The resulting clotting times are converted to units/ml, based on the same human standard curve described above. See, for example, U.S. Pat. No. 6,376,463.

The factor VIII_{SEP} polypeptides may be altered in various ways including amino acid substitutions, deletions, truncations, and insertions. Methods for such manipulations are generally known in the art. For example, amino acid sequence variants of the factor VIII_{SEP} proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity (i.e., high-level expression) of the factor VIII_{SEP} may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred. Alternatively, methods to minimize the number of porcine amino acids in the A₁ and ap-A₃ domains of factor VIII_{SEP} and still continue to retain the high-level expression of the factor VIII_{SEP} are known in the art and include, for example, established site-directed mutagenesis such as by splicing overlap extension as described elsewhere herein. Obviously, the mutations that will be made in the DNA encoding the variant must not place the sequence out of reading frame and preferably will not create complementary regions that could produce secondary mRNA structure. See, EP Patent Application Publication No. 75,444.

When it is difficult to predict the exact effect of the substitution, deletion, or insertion in advance of doing so, one skilled in the art will appreciate that the effect will be evaluated by routine screening assays. That is, the activity can be evaluated by high-level expression of the factor VIII polypeptide as discussed in detail elsewhere herein.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5:Suppl. 3 (National Biomedical Research Foundation, Washington, D.C.). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences is accomplished using a mathematical algorithm. Specifically, for the purpose of the present invention percent identity of an amino acid sequence is determined using the Smith-Waterman homology search algorithm using an affine 6 gap search with a gap open penalty of 12 5 and a gap extension penalty of 2, BLOSUM matrix 62. The Smith-Waterman homology search algorithm is taught in Smith and Waterman (1981) Adv. Appl. Math 2:482-489, herein incorporated by reference. Alternatively, for the purposes of the present invention percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

It is further recognized that when considering percentage of amino acid identity, some amino acid positions may differ as a result of conservative amino acid substitutions, which do not effect the properties of polynucleotide function. In these instances, percent sequence identity may be adjusted upwards to account for the similarity in conservatively substituted amino acids. Such adjustments are well known in the art. See, for example, Meyers et al. (1988) Computer Apphc. Bioi. Sci. 4:11-17.

It is recognized that the variant factor VIII_{SEP} or fragments thereof can be made (1) by substitution of isolated, plasma-derived animal subunits or human subunits (heavy or light chains) for corresponding human subunits or animal subunits; (2) by substitution of human domains or animal domains (A1, A2, A3, B, C1, and C2) for corresponding animal domains or human domains; (3) by substitution of parts of human domains or animal domains for parts of animal domains or human domains; (4) by substitution of at least one specific sequence including one or more unique human or animal amino acid(s) for the corresponding animal or human amino acid(s); or (5) by substitution of amino acid sequence that has no known sequence identity to factor VIII for at least one sequence including one or more specific amino acid residue(s) in human, animal, or variant factor VIII or fragments thereof. Individual amino acid replacements can be obtain by site-directed mutagenesis of the corresponding segment coding DNA.

In a factor VIII molecule, a “domain”, as used herein, is a continuous sequence of amino acids that is defined by internal amino acid sequence identity and sites of proteolytic cleavage by thrombin. Unless otherwise specified, factor VIII domains include the following amino acid residues, when the sequences are aligned with the human amino acid sequence (SEQ ID NO:6): A1, residues A1a1-Arg372; A2, residues Ser373-Arg740; B, residues Ser741-Arg1648; A3, residues Ser1690-Ile2032; C1, residues Arg2033-Asn2172; C2, residues Ser2173-Tyr2332. The A3-C1-C2 sequence includes residues Ser1690-Tyr2332. The remaining sequence, residues Glu1649Arg1689, is usually referred to as the factor VIII light chain activation peptide. Factor VIII is proteolytically activated by thrombin or factor Xa, which dissociates it from von Willebrand factor, forming factor VIII, which has procoagulant function. The biological function of factor VIIIa is to increase the catalytic efficiency of factor 1Xa toward factor X activation by several orders of magnitude. Thrombin-activated factor VIIIa is a 160 kDa A1/A2/A3-C1-C2 heterotrimer that forms a complex with factor IXa and factor X on the surface of platelets or monocytes. A “partial domain” as used herein is a continuous sequence of amino acids forming part of a domain. “Subunits” of human or animal (i.e., mouse, pig, dog etc.) factor VIII, as used herein, are the heavy and light chains of the protein. The heavy chain of factor VIII contains three domains, A1, A2, and B. The light chain of factor VIII also contains three domains, A3, C1, and C2. A “unique” amino acid residue or sequence, as used herein, refers to an amino acid sequence or residue in the factor VIII molecule of one species that is different from the homologous residue or sequence in the factor VIII molecule of another species. As used herein, “mammalian factor VIII” includes factor VIII with amino acid sequence derived from any non-human mammal, unless otherwise specified. “Animal”, as used herein, refers to pig and other non-human mammals.

Since current information indicates that the B domain has no inhibitory epitope and has no known effect on factor VIII function, factor VIII_{SEP} variants of the present invention may have a B domain or a portion thereof. In addition, factor VIII_{SEP} variants may also have the factor VIII B-domain with the B-domain from porcine or human factor V. See, for example, U.S. Pat. No. 5,004,803. A “B-domainless” variant factor VIII_{SEP} or fragment thereof, as used herein, refers to any one of the variant factor VIII_{SEP} constructs described herein that lacks the B domain, or a portion thereof.

One of skill in the art will be aware of techniques that allow individual subunits, domains, or continuous parts of domains of animal or human factor VIII cDNA to be cloned and substituted for the corresponding human or porcine subunits, domains, or parts of domains by established mutagenesis techniques and thereby generate a factor VIII_{SEP} or variant or fragment thereof. For example, Lubin et al. (1994) J Biol. Chem. 269(12):8639-8641 describes techniques for substituting the porcine A2 domain for the human domain using convenient restriction sites. Other methods for substituting a region of the factor VIII cDNA of one species for the factor VIII cDNA of another species include splicing by overlap extension (“SOE”), as described by Horton et al. (1993) Meth. Enzymol 217:270-279.

### DNA Constructs and Vectors

The nucleotide sequence encoding the factor VIII_{SEP} polypeptides or active variants or fragments thereof can be contained in a DNA construct. The DNA construct can include a variety of enhancers/promoters from both viral and mammalian sources that drive expression of the factor VIII_{SEP} polypeptide in the desired cell type. The DNA construct can further contain 3′ regulatory sequences and nucleic acid sequences that facilitate subcloning and recovery of the DNA.

The transcriptional promoter and, if desired, the transcriptional enhancer element are operably linked to the nucleic acid sequence of the factor VIII polypeptide. A “promoter” is defined as a minimal DNA sequence that is sufficient to direct transcription of a nucleic acid sequence. A “transcriptional enhancer element” refers to a regulatory DNA sequence that stimulates the transcription of the adjacent gene. The nucleic acid sequence encoding the factor VIII polypeptide is operably linked to the promoter sequence. See, for example, Goeddel (1990) Gene Expression Technology: Methods in Enzymology 185 (Academic Press, San Diego, CA).

By “operably linked” is intended a functional linkage between the regulatory promoter and the nucleic acid sequence encoding the factor VIII polypeptide. The functional linkage permits gene expression of factor VIII when the appropriate transcription activator proteins are present.

Thus, the DNA construct can include a promoter that may be native or foreign. By “foreign” it is meant a sequence not found in the native organism. Furthermore, the transcription regulatory elements may be heterologous to the nucleotide sequence encoding factor VIII. By “heterologous” is intended any nucleotide sequence not naturally found upstream of the sequence encoding the factor VIII polypeptide. The promoter may be a natural sequence or a synthetic sequence. In addition, the promoter may be constitutively active, tissue-specific, or inducible. A tissue-specific promoter is preferentially activated in a given tissue and results in expression of a gene product in the tissue where activated.

For use in mammalian cells, the promoters may be derived from a virus. For example, commonly used promoters are derived from polyoma, Simian Virus 40 (SV40) and Adenovirus 2. The early and late promoters of SV40 virus are useful as is the major late promoter of adenovirus. Further, it is also possible, and often desirable, to utilize promoter or control sequences normally associated with the desired gene sequence, provided such control sequences are compatible with the host cell system.

In certain variations, the introduction of the nucleotide sequence encoding factor VIII into a cell can be identified in vitro or in vivo by including a marker in the DNA construct. The marker will result in an identifiable change in the genetically transformed cell. Drug selection markers include for example neomycin, puromycin, hygromycin, DHFR, GPT, zeocin and histidinol. Alternatively, enzymes such as herpes simplex virus thymidine kinase (TK) or immunological markers can be used. Further examples of selectable markers are well known in the art.

It is recognized that multiple alterations can be envisioned for the design of the DNA construct used in the methods of the present invention. For instance, the construct may be designed for the insertion of the nucleotide sequence encoding the factor VIII_{SEP} polypeptide using homologous or site-specific recombination systems (i.e., ere or FLP recombination systems).

The DNA construct may also contain at least one additional gene to be co-introduced into the host cells.

The nucleotide sequences can be contained in an expression vector. An “expression vector” is a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art.

Other vectors, including both plasmid and eukaryotic viral vectors, may be used to express a recombinant gene construct in eukaryotic cells depending on the preference and judgment of the skilled practitioner (see, for example, Sambrook et al., Chapter 16). For example, many viral vectors are known in the art including, for example, retroviruses, adeno-associated viruses, and adenoviruses. Other viruses useful for introduction of a gene into a cell include, but a not limited to, herpes virus, mumps virus, poliovirus, Sindbis virus, and vaccinia virus, such as, canary pox virus. The methods for producing replication-deficient viral particles and for manipulating the viral genomes are well known. See, for examples, Rosenfeld et al. (1991) Science 252:431-434, Rosenfeld et al. (1992) Cell 68:143-155, and U.S. Pat. No. 5,882,877 (adenovirus); U.S. Pat. No. 5,139,941 (adeno-associated virus); U.S. Pat. Nos. 4,861,719, 5,681,746, and Miller et al. (1993) Methods in Enzymology 217:581 (retrovirus), all of which are herein incorporated by reference. Therefore, given the knowledge in the art, viral vectors can be readily constructed for use in the introduction of the factor VIII sequences into a cell. Other vectors and expression systems, including bacterial, yeast, and insect cell systems, can be used but are not preferred due to differences in, or lack of, glycosylation.

Factor VIII polypeptides can be expressed in a variety of cells commonly used for culture and recombinant mammalian protein expression. In particular, a number of rodent cell lines have been found to be especially useful hosts for expression of large proteins. Preferred cell lines, available from the American Type Culture Collection, Rockville, Md., include, but are not limited to, baby hamster kidney cells, and chinese hamster ovary (CHO) cells which are cultured using routine procedures and media. Additional cells of interest can include vertebrate cells such as VERO, HeLa cells, W138, COS-7, and MDCK cell lines. For other suitable expression systems see chapters 16 and 17 of Sambrook et al. (1989) Molecular cloning: A Laboratory Manual (2d ed., Cold Spring Harbor Laboratory Press, Plainview, NY). See, Goeddel (1990) in Gene Expression Technology: Methods in Enzymology 185 (Academic Press, San Diego, CA).

### Methods of Expression and Isolation

The DNA construct may be introduced into a cell (prokaryotic or eukaryotic) by standard methods. As used herein, the terms “transformation” and “transfection” are intended to refer to a variety of art recognized techniques to introduce a DNA into a host cell. Such methods include, for example, transfection, including, but not limited to, liposome-polybrene, DEAE dextranmediated transfection, electroporation, calcium phosphate precipitation, microinjection, or velocity driven microprojectiles (“biolistics”). Such techniques are well known by one skilled in the art. See, Sambrook et al. (1989) Molecular Cloning: A Laboratory Manaual (2 ed. Cold Spring Harbor Lab Press, Plainview, NY). Alternatively, one could use a system that delivers the DNA construct in a gene delivery vehicle. The gene delivery vehicle may be viral or chemical. Various viral gene delivery vehicles can be used with the present invention. In general, viral vectors are composed of viral particles derived from naturally occurring viruses. The naturally occurring virus has been genetically modified to be replication defective and does not generate additional infectious viruses. The viral vector also contains a DNA construct capable of expressing the factor VIII protein.

The DNA construct containing nucleic acid sequences encoding the factor VIII_{SEP} polypeptide may also be administered to cell by a non-viral gene delivery vehicle. Such chemical gene delivery vehicles include, for example, a DNA- or RNA-liposome complex formulation or a naked DNA. See, for example, Wang et al. (1987) Proc. Natl. Acad. Sci. U.S.A. 84:7851, U.S. Pat. Nos. 5,844,107, 5,108,921, and Wagner et al. (1991) Proc. Natl. Acad. Sci. U.S.A. 88:4255-4259, all of which are herein incorporated by reference.

It is recognized that the method of introducing the factor VIII_{SEP} polypeptide or variant or fragment thereof into a cell can result in either stable integration into the cell genome or transient, episomal expression.

As defined herein, the “expression product” of a DNA encoding a factor VIII_{SEP} polypeptide or a fragment or variant thereof is the product obtained from expression of the referenced DNA in a suitable host cell, including such features of pre- or post-translational modification of protein encoded by the referenced DNA, including but not limited to glycosylation, proteolytic cleavage and the like. It is known in the art that such modifications can occur and can differ somewhat depending upon host cell type and other factors, and can result in molecular isoforms of the product, with retention of procoagulant activity. See, for example, Lind et al, (1995) Eur. J. Biochem. 232:1927 incorporated herein by reference.

In a one variation, cDNA encoding factor VIII_{SEP} or a variant or fragment thereof, is inserted in a mammalian expression vector, such as ReNeo. Preliminary characterization of the factor VIII_{SEP} is accomplished by transient expression in the ReNeo expression vector containing the factor VIII_{SEP} construct in COS-7 cells. A determination of whether active factor VIII_{SEP} protein is expressed can then be made. The expression vector construct is used further to stably transfect cells in culture, such as baby hamster kidney cells, using methods that are routine in the art, such as liposome-mediated transfection (Lipofectin™, Life Technologies, Inc.). Expression of the factor VIII_{SEP} protein can be confirmed, for example, by sequencing, Northern and Western blotting, or polymerase chain reaction (PCR).

Factor VIII_{SEP} polypeptides or fragments or variants thereof in the culture media in which the transfected cells stably expressing the protein are maintained can be precipitated, pelleted, washed, and resuspended in an appropriate buffer, and the factor VIII_{SEP} protein or variant or fragment thereof is purified by standard techniques, including immunoaffinity chromatography using, for example, monoclonal anti-A2-Sepharose™.

A “fusion protein” or “fusion factor VIII_{SEP} or fragment thereof”, as used herein, is the product of a hybrid gene in which the coding sequence for one protein is extensively altered, for example, by fusing part of it to the coding sequence for a second protein from a different gene to produce a hybrid gene that encodes the fusion protein.

In a further embodiment, the factor VIII_{SEP} or variant or fragment thereof is expressed as a fusion protein from a recombinant molecule in which sequence encoding a protein or peptide that enhances, for example, stability, secretion, detection, isolation, or the like is inserted in place adjacent to the factor VIII encoding sequence. See, for example, U.S. Pat. No. 4,965,199 which discloses a recombinant DNA method for producing factor VIII in mammalian host cells and purification of human factor VIII. Human factor VIII expression on CRG (Chinese hamster ovary) cells and BHKC (baby hamster kidney cells) has been reported. Established protocols for use of homologous or heterologous species expression control sequences including, for example, promoters, operators, and regulators, in the preparation of fusion proteins are known and routinely used in the art. See, Ausubel et al. Current Protocols in Molecular Biology, Wiley Interscience, N.Y, herein incorporated by reference. It is further noted that expression is enhanced by including portions of the B-domain. In particular, the inclusion of those parts of the B domain designated “SQ” (Lind et al. (1995) Eur. J. Biochem. 232:1927, herein incorporated herein by reference) results in favorable expression. “SQ” constructs lack all of the human B domain except for 5 amino acids of the B domain N-terminus and 9 amino acids of the B domain C-terminus.

It is further recognized that the factor VIII_{SEP} polypeptide or variant or fragment thereof may be prepared via reconstitution methods. In this variation factor VIII_{SEP}, variants or fragments thereof are made by isolation of subunits, domains, or continuous parts of domains of plasma-derived factor VIII, followed by reconstitution and purification to produce a factor VIII_{SEP} polypeptide of the invention. Alternatively, the factor VIII_{SEP}, variant or fragment thereof can be made by recombinant DNA methods, followed by reconstitution and purification.

More particularly, the method of preparing a factor VIII_{SEP} by reconstitution methods can be performed via a modification of procedures reported by Fay et al. (1990) J. Biol. Chem. 265:6197; and Lollar et al. (1988) J. Biol. Chem. 263:10451, which involves the isolation of subunits (heavy and light chains) of human and animal factor VIII, followed by recombination of human heavy chain and animal light chain or by recombination of human light chain and animal heavy chain.

Isolation of both human and animal individual subunits involves dissociation of the light chain/heavy chain dimer. This is accomplished, for example, by chelation of calcium with ethylenediaminetetraacetic acid (EDTA), followed by monoS™ HPLC (Pharmacia-LKB, Piscataway, N.J.). Hybrid human/animal factor VIII molecules are reconstituted from isolated subunits in the presence of calcium. Hybrid human light chain/animal heavy chain or animal light chain/human heavy chain factor VIII is isolated from unreacted heavy chains by monoS™ HPLC by procedures for the isolation of porcine factor VIII, such as described by Lollar et al. (1988) Blood 71:137-143 and in U.S. Pat. No. 6,376,463, both of which is herein incorporated by reference.

### Diagnostic Assays

As used herein, “diagnostic assays” include assays that in some manner utilize the antigen-antibody interaction to detect and/or quantify the amount of a particular antibody that is present in a test sample to assist in the selection of medical therapies. There are many such assays known to those of skill in the art. As used herein, however, the factor VIII_{SEP} DNA or variant or fragment thereof and protein expressed therefrom, in whole or in part, can be substituted for the corresponding reagents in the otherwise known assays, whereby the modified assays may be used to detect and/or quantify antibodies to factor VIII. It is the use of these reagents, the factor VIII_{SEP} DNA or variants or fragments thereof or protein expressed therefrom, that permits modification of known assays for detection of antibodies to human or animal factor VIII or to hybrid human/animal factor VIII. As used herein, the factor VIII_{SEP} or variants or fragment thereof that includes at least one epitope of the protein can be used as the diagnostic reagent.

The DNA or amino acid sequence of the factor VIII_{SEP} or variant or fragment thereof can be used in assays as diagnostic reagents for the detection of inhibitory antibodies to human or animal factor VIII, including, for example, samples of serum and body fluids of human patients with factor VIII deficiency. These antibody assays include assays such as ELISA assays, immunoblots, radioimmunoassays, immunodiffusion assays, and assay of factor VIII biological activity (e.g., by coagulation assay). Examples of other assays in which the factor VIII_{SEP} or variant or fragment thereof can be used include the Bethesda assay and anticoagulation assays.

Techniques for preparing these reagents and methods for use thereof are known to those skilled in the art. For example, an immunoassay for detection of inhibitory antibodies in a patient serum sample can include reacting the test sample with a sufficient amount of the factor VIII_{SEP} that contains at least one antigenic site, wherein the amount is sufficient to form a detectable complex with the inhibitory antibodies in the sample.

Nucleic acid and amino acid probes can be prepared based on the sequence of the factor VIII_{SEP} DNA or protein molecule or fragments or variants thereof. In some variations, these can be labeled using dyes or enzymatic, fluorescent, chemiluminescent, or radioactive labels that are commercially available. The amino acid probes can be used, for example, to screen sera or other body fluids where the presence of inhibitors to human, animal, or hybrid human/animal factor VIII is suspected. Levels of inhibitors can be quantitated in patients and compared to healthy controls, and can be used, for example, to determine whether a patient with a factor VIII deficiency can be treated with a factor VIII_{SEP} or active fragment or variant thereof. The cDNA probes can be used, for example, for research purposes in screening DNA libraries.

### Pharmaceutical Compositions

We further provide pharmaceutical compositions comprising the nucleic acid molecules and the polypeptides encoding the high-level expression factor VIII_{SEP} or variants and fragments thereof. Such compositions can comprise nucleic acids and polypeptides of the invention either alone or in combination with appropriate pharmaceutical stabilization compounds, delivery vehicles, and/or carrier vehicles, are prepared according to known methods, as described in Martin et al. Remington's Pharmaceutical Sciences, herein incorporated by reference.

In one variation, the carriers or delivery vehicles for intravenous infusion are physiological saline or phosphate buffered saline.

In another embodiment, suitable stabilization compounds, delivery vehicles, and carrier vehicles include but are not limited to other human or animal proteins such as albumin.

Phospholipid vesicles or liposomal suspensions may also be used as pharmaceutically acceptable carriers or delivery vehicles. These can be prepared according to methods known to those skilled in the art and can contain, for example, phosphatidylserine-phosphatidylcholine or other compositions of phospholipids or detergents that together impart a negative charge to the surface, since factor VIII binds to negatively charged phospholipid membranes. Liposomes may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the factor VIII_{SEP} of the present invention is then introduced into the container. The container is then swirled by hand to free lipid material from the sides of the container and to disperse lipid aggregates, thereby forming the liposomal suspension.

The factor VIII_{SEP} molecules can be combined with other suitable stabilization compounds, delivery vehicles, and/or carrier vehicles, including vitamin K dependent clotting factors, tissue factor, and von Willebrand factor (vWf) or a fragment of vWf that contains the factor VIII binding site, and polysaccharides such as sucrose.

Factor VIII_{SEP} molecules can also be delivered by gene therapy using delivery means such as retroviral vectors. This method consists of incorporation of a nucleotide sequence encoding desired factor VIII_{SEP} polypeptide into human cells that are transplanted directly into a factor VIII_{SEP} deficient patient or that are placed in an implantable device, permeable to the factor VIII molecules but impermeable to cells, that is then transplanted.

In one variation, the method will be retroviral-mediated gene transfer. In this method, a nucleotide sequence encoding a factor VIII polypeptide is cloned into the genome of a modified retrovirus. The gene is inserted into the genome of the host cell by viral machinery where it will be expressed by the cell. The retroviral vector is modified so that it will not produce virus, preventing viral infection of the host. The general principles for this type of therapy are known to those skilled in the art and have been reviewed in the literature (Kohn et al. (1989) Transfusion 29:812-820).

The factor VIII_{SEP} polypeptide can be stored bound to vWf to increase the half-life and shelf-life of the polypeptide molecule. Additionally, lyophilization of factor VIII_{SEP} can improve the yields of active molecules in the presence of vWf. Current methods for storage of human and animal factor VIII used by commercial suppliers can be employed for storage of recombinant factor VIII. These methods include: (1) lyophilization of factor VIII_{SEP} in a partially-purified state (as a factor VIII “concentrate” that is infused without further purification); (2) immunoaffinity-purification of factor VIII_{SEP} by the Zimmerman method and lyophilization in the presence of albumin, which stabilizes the factor VIII; (3) lyophilization of recombinant factor VIII_{SEP} in the presence of albumin.

Additionally, the factor VIII polypeptides can be stored at 4° C. in 0.6 M NaCl, mM MES, and 5 mM CaCl₂) at pH 6.0. The polypeptides can also be stored frozen in these buffers and thawed with minimal loss of activity.

### Methods of Treatment

Factor VIII_{SEP} or fragments and variant thereof can be used to treat uncontrolled bleeding due to factor VIII deficiency (e.g., intraarticular, intracranial, or gastrointestinal hemorrhage) in hemophiliacs with and without inhibitory antibodies and in patients with acquired factor VIII deficiency due to the development of inhibitory antibodies. The active materials are preferably administered intravenously.

“Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

Additionally, factor VIII_{SEP} or fragments and variant thereof can be administered by transplantation of cells genetically engineered to produce the factor VIII_{SEP} or by implantation of a device containing such cells, as described above.

In one variation, pharmaceutical compositions of factor VIII_{SEP} or fragments and variants thereof alone or in combination with stabilizers, delivery vehicles, and/or carriers are infused into patients intravenously according to the same procedure that is used for infusion of factor VIII_{SEP}.

The treatment dosages of the factor VIII_{SEP} composition or variants or fragments thereof that must be administered to a patient in need of such treatment will vary depending on the severity of the factor VIII deficiency. Generally, dosage level is adjusted in frequency, duration, and units in keeping with the severity and duration of each patient's bleeding episode. Accordingly, the factor VIII_{SEP} or variants or fragments thereof is included in the pharmaceutically acceptable carrier, delivery vehicle, or stabilizer in an amount sufficient to deliver to a patient a therapeutically effective amount of the hybrid to stop bleeding, as measured by standard clotting assays.

“Specific activity” as used herein, refers to the activity that will correct the coagulation defect of human factor VIII deficient plasma. Specific activity is measured in units of clotting activity per milligram total factor VIII protein in a standard assay in which the clotting time of human factor VIII deficient plasma is compared to that of normal human plasma. One unit of factor VIII activity is the activity present in one milliliter of normal human plasma. In the assay, the shorter the time for clot formation, the greater the activity of the factor VIII being assayed. The specific activity of the factor VIII polypeptides, variant or fragments thereof, may be less than, equal to, or greater than that of either plasma-derived or recombinant human factor VIII.

Factor VIII is classically defined as that substance present in normal blood plasma that corrects the clotting defect in plasma derived from individuals with hemophilia A. The coagulant activity in vitro of purified and partially-purified forms of factor VIII_{SEP} is used to calculate the dose of factor VIII for infusions in human patients and is a reliable indicator of activity recovered from patient plasma and of correction of the in vivo bleeding defect. There are no reported discrepancies between standard assay of novel factor VIII molecules in vitro and their behavior in the dog infusion model or in human patients, according to Lusher et al. New Engl. J. Med. 328:453-459; Pittman et al. (1992) Blood 79:389-397; and Brinkhous et al. (1985) Proc. Natl. Acad. Sci. 82:8752-8755.

The increase of factor VIII_{SEP} in the plasma will be sufficient to produce a therapeutic effect. A “therapeutic effect” is defined as an increase in the blood coagulation activity in the plasma of patients that is greater than the coagulation activity observed in the subject before administration of the factor VIII_{SEP} molecule. In a standard blood clotting assay, the shorter time for clot formation, the greater the activity of factor VIII being assayed. An increase in factor VIII activity in the factor VIII deficient plasma of at least 1% or higher will be therapeutically beneficial.

Usually, the desired plasma factor VIII level to be achieved in the patient through administration of the factor VIII_{SEP} or variant or fragment thereof is in the range of 30-100% of normal. In a one mode of administration of the factor VIII_{SEP} or fragment or variant thereof, the composition is given intravenously at a preferred dosage in the range from about 5 to 50 units/kg body weight, more preferably in a range of 10-50 units/kg body weight, and most preferably at a dosage of 20-40 units/kg body weight; the interval frequency is in the range from about 8 to 24 hours (in severely affected hemophiliacs); and the duration of treatment in days is in the range from 1 to 10 days or until the bleeding episode is resolved. See, for example, Roberts et al. (1990) Hematology, Williams et al. ed. Ch. 153, 1453-1474, herein incorporated by reference. Patients with inhibitors may require more factor VIII_{SEP} or variants or fragments thereof, or patients may require less factor VIII_{SEP} or fragments or variants thereof. As in treatment with human or porcine factor VIII, the amount of factor VIII_{SEP} or fragments or variants infused is defamed by the one-stage factor VIII coagulation assay and, in selected instances, in vivo recovery is determined by measuring the factor VIII in the patient's plasma after infusion. It is to be understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

Treatment can take the form of a single intravenous administration of the composition or periodic or continuous administration over an extended period of time, as required. Alternatively, factor VIII_{SEP} or fragments or variants thereof can be administered subcutaneously or orally with liposomes in one or several doses at varying intervals of time.

Factor VIII_{SEP} or fragments or variants thereof can also be used to treat uncontrolled bleeding due to factor VIII deficiency in hemophiliacs who have developed antibodies to human factor VIII.

### EXPERIMENTAL

### Example 1

### Sequence Characterization of Factor VIII

Both porcine and human factor VIII are isolated from plasma as a two subunit protein. The subunits, known as the heavy chain and light chain, are held together by a non-covalent bond that requires calcium or other divalent metal ions. The heavy chain of factor VIII contains three domains, A1, A2, and B, which are linked covalently. The light chain of factor VIII also contains three domains, designated A3, C1, and C2. The B domain has no known biological function and can be removed, or partially removed from the molecule proteolytically or by recombinant DNA technology methods without significant alteration in any measurable parameter of factor VIII. Human recombinant factor VIII has a similar structure and function to plasma-derived factor VIII, though it is not glycosylated unless expressed in mammalian cells. Both human and porcine activated factor VIII (“factor VIIIa”) have three subunits due to cleavage of the heavy chain between the A1 and A2 domains. This structure is designated A1/A2/A3-C1-C2.

The cDNA sequence of porcine factor VIII corresponding the signal peptide coding region, the A1, B, light chain activity peptide region A3, C1, and C2 domains is provided in SEQ ID NO:1. The translation of the porcine cDNA is provided in SEQ ID NO:2.

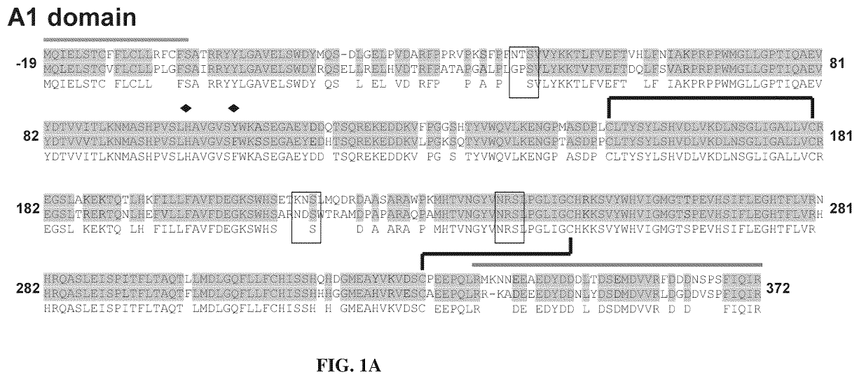

Potential N-linked glycosylation sites (NXS/T where X is not proline) can be seen in FIGS. 1A-1B. There are eight conserved N-linked glycosylation sites: one in the A1 domain, one in the A2 domain, four in the B domain, one in the A3 domain, and one in the C1 domain. The 19 A and C domain cysteines are conserved, whereas there is divergence of B domain cysteines. Six of the seven disulfide linkages in factor VIII are found at homologous sites in factor V and Ceruloplasmin, and both C domain disulfide linkages are found in factor V (McMullen et al. (1995) Protein Sci. 4:740-746). Human factor VIII contains sulfated tyrosines at positions 346, 718, 719, 723, 1664, and 1680 (Pittman et al. (1992) Biochemistry 31:3315-3325; Michnick et al. (1994) J. Biol. Chem. 269:20095-20102). These residues are conserved in mouse factor VIII and porcine factor VIII (FIGS. 1A and 1B), although the CLUSTALW program failed to align the mouse tyrosine corresponding to Tyr346 in human factor VIII. Epitopes of the various domain of the factor VIII polypeptide are outlined in FIGS. 1A and 1B.

### Example 2

### Summary

Human factor VIII expression levels are significantly lower than levels of other coagulation proteins in vivo and in heterologous expression systems in vitro. Low-level expression of recombinant human factor VIII has constrained the treatment of hemophilia A using recombinant protein infusion and gene therapy protocols. However, recombinant B-domain-deleted porcine factor VIII is expressed at levels 10-14 fold greater than recombinant B-domain-deleted human factor VIII in vitro. To identify sequences of porcine factor VIII necessary for this property, B-domain-deleted human/porcine hybrid factor VIII cDNAs were produced that contained substitution of human sequences with the corresponding porcine sequences. These cDNAs were transiently transfected into COS-7 cells or stably transfected into BHK-derived cells and factor VIII expression into the extracellular media was measured by one-stage coagulation assay. Human/porcine hybrid factor VIII cDNAs containing 1) the A1, A2 and A3 domains of porcine factor VIII and the C1 and C2 domains of human factor VIII, or 2) the A1 and A3 domains of porcine factor VIII and the A2, C1, and C2 domains of human factor VIII demonstrated factor VIII expression levels comparable to porcine factor VIII. A human/porcine hybrid factor VIII molecule demonstrating high-level expression may be valuable for improving factor VIII production for intravenous infusion or for somatic cell gene therapy of hemophilia A.

### Materials

Dulbecco's phosphate-buffered saline, fetal bovine serum (FBS), penicillin, streptomycin, DMEM:F12, serum-free AIM V culture media, Lipofectin, Lipofectamine 2000 and geneticin were purchased from Invitrogen. Baby hamster kidney-derived cells, designated BHK-M cells (Funk et al. (1990) Biochemistry 29:1654-1660), were a gift from Dr. Ross Macgillivray, University of British Columbia. Transient transfections were controlled for transfection efficiency using the RL-CMV vector and Dual-Luciferase Assay Kit (Promega, Madison, WI). Citrated factor VIII-deficient plasma and pooled citrated normal human plasma (FACT) were purchased from George King Biomedical (Overland Park, KA). Activated partial thromboplastin reagent (aPTT) was purchased from Organon Teknika (Durham, NC). Oligonucleotide primers were synthesized by Life Technologies. Pfu DNA polymerase and E. coli XL-1 Blue cells were purchased from Stratagene (La Jolla, CA).

### Construction of Factor VIII Expression Vectors

All of the factor VIII expression vectors in this study were contained in the ReNeo mammalian expression plasmid (Lind et al. (1995) Eur. J. Biochem. 232: 1927). The factor VIII cDNA inserts lack endogenous factor VIII 5′-UTR sequence and contain the first 749 of the 1805 nt human factor VIII 3′-UTR.

A human B domain-deleted factor VIII cDNA designed HSQ (SEQ ID NO: 28) was created by cloning the human factor VIII cDNA into the mammalian expression vector ReNeo as described previously (Doering et al. (2002) J. Biol. Chem. 277: 38345-38349). The HSQ cDNA encodes an SFS Q N P P V L K R H Q R (SEQ ID NO:9) linker sequence between the A2 and ap domains. This linker includes the R H Q R (SEQ ID NO:10) recognition sequence for intracellular proteolytic processing by PACE/furin (Seidah et al. (1997) Current Opinion in Biotechnology 8:602-607). This cleavage event converts single chain factor VIII into a heterodimer (Lind et al. (1995) Eur. J. Biochem. 232:19-27). Heterodimeric factor VIII is considered the physiologic form of factor VIII (Fass et al. (1982) Blood 59:594-600).

ETX-A1-FV (SEQ ID NO: 15), which contains the A1 domain of HSQ (SEQ ID NO:28) with 10 amino acid substitutions.

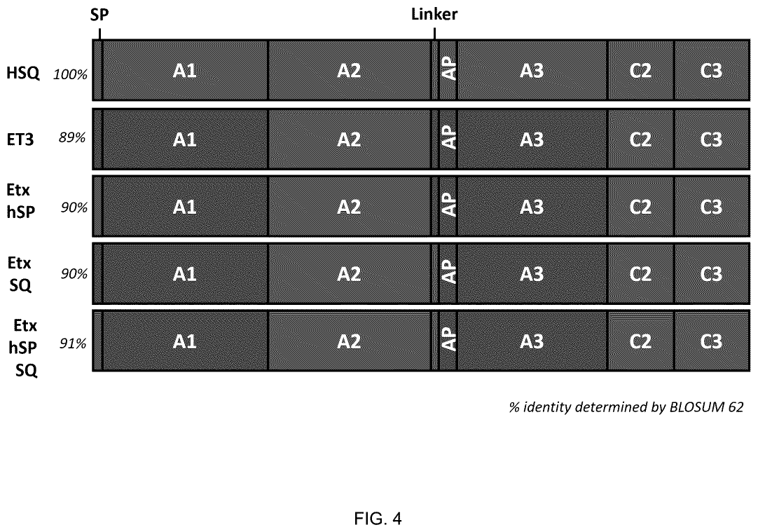

ETX-A1-FV A1 Domain Sequence
(SEQ ID NO: 23)
ATRRYYLGAVELSWDYRQSDLGELPVDARFPPRVPKSFPFNTSVVYKKTVFVEFTD
HLFNIAKPRPPWMGLLGPTIQAEVYDTVVVTLKNMASHPVSLHAVGVSYWKASEG
AEYDDHTSQREKEDDKVFPGGSHTYVWQVLKENGPTASDPPCLTYSYLSHVDLVK
DLNSGLIGALLVCREGSLTKEKTQTLHKFVLLFAVFDEGKSWHSETKNSLMQDRDA
ASARAWPKMHTVNGYVNRSLPGLIGCHRKSVYWHVIGMGTTPEVHSIFLEGHTFLV
RHHRQASLEISPITFLTAQTLLMDLGQFLLFCHISSHQHDGMEAYVKVDSCPEEPQLR
MKANEEAEDYDDDLTDSEMDVVRFDDDNSPSFIQIR
ETX-A1-FV Full fVIII Sequence
(SEQ ID NO: 15)
MQLELSTCVFLCLLPLGFSATRRYYLGAVELSWDYRQSDLGELPVDARFP
PRVPKSFPFNTSVVYKKTVFVEFTDHLFNIAKPRPPWMGLLGPTIQAEVYDTVVVTL
KNMASHPVSLHAVGVSYWKASEGAEYDDHTSQREKEDDKVFPGGSHTYVWQVLK
ENGPTASDPPCLTYSYLSHVDLVKDLNSGLIGALLVCREGSLTKEKTQTLHKFVLLF
AVFDEGKSWHSETKNSLMQDRDAASARAWPKMHTVNGYVNRSLPGLIGCHRKSV
YWHVIGMGTTPEVHSIFLEGHTFLVRHHRQASLEISPITFLTAQTLLMDLGQFLLFCHI
SSHQHDGMEAYVKVDSCPEEPQLRMKANEEAEDYDDDLTDSEMDVVRFDDDNSPS
FIQIRSVAKKHPKTWVHYIAAEEEDWDYAPLVLAPDDRSYKSQYLNNGPQRIGRKY
KKVRFMAYTDETFKTREAIQHESGILGPLLYGEVGDTLLIIFKNQASRPYNIYPHGITD
VRPLYSRRLPKGVKHLKDFPILPGEIFKYKWTVTVEDGPTKSDPRCLTRYYSSFVNM
ERDLASGLIGPLLICYKESVDQRGNQIMSDKRNVILFSVFDENRSWYLTENIQRFLPN
PAGVQLEDPEFQASNIMHSINGYVFDSLQLSVCLHEVAYWYILSIGAQTDFLSVFFSG
YTFKHKMVYEDTLTLFPFSGETVFMSMENPGLWILGCHNSDFRNRGMTALLKVSSC
DKNTGDYYEDSYEDISAYLLSKNNAIEPRSFAQNSRPPSASAPKPPVLRRHQRDISLP
TFQPEEDKMDYDDIFSTETKGEDFDIYGEDENQDPRSFQKRTRHYFIAAVEQLWDYG
MSESPRALRNRAQNGEVPRFKKVVFREFADGSFTQPSYRGELNKHLGLLGPYIRAEV
EDNIMVTFKNQASRPYSFYSSLISYPDDQEQGAEPRHNFVQPNETRTYFWKVQHHM
APTEDEFDCKAWAYFSDVDLEKDVHSGLIGPLLICRANTLNAAHGRQVTVQEFALF
FTIFDETKSWYFTENVERNCRAPCHLQMEDPTLKENYRFHAINGYVMDTLPGLVMA
QNQRIRWYLLSMGSNENIHSIHFSGHVFSVRKKEEYKMAVYNLYPGVFETVEMLPS
KVGIWRIECLIGEHLQAGMSTTFLVYSKKCQTPLGMASGHIRDFQITASGQYGQWAP
KLARLHYSGSINAWSTKEPFSWIKVDLLAPMIIHGIKTQGARQKFSSLYISQFIIMYSL
DGKKWQTYRGNSTGTLMVFFGNVDSSGIKHNIFNPPIIARYIRLHPTHYSIRSTLRME
LMGCDLNSCSMPLGMESKAISDAQITASSYFTNMFATWSPSKARLHLQGRSNAWRP
QVNNPKEWLQVDFQKTMKVTGVTTQGVKSLLTSMYVKEFLISSSQDGHQWTLFFQ
NGKVKVFQGNQDSFTPVVNSLDPPLLTRY
LRIHPQSWVHQIALRMEVLGCEAQDLY
EXT-A3-Cu
(SEQ ID NO: 16)
MQLELSTCVFLCLLPLGFSAIRRYYLGAVELSWDYRQSELLRELHVDTRFPATAPGA
LPLGPSVLYKKTVFVEFTDQLFSVARPRPPWMGLLGPTIQAEVYDTVVVTLKNMAS
HPVSLHAVGVSFWKSSEGAEYEDHTSQREKEDDKVLPGKSQTYVWQVLKENGPTA
SDPPCLTYSYLSHVDLVKDLNSGLIGALLVCREGSLTRERTQNLHEFVLLFAVFDEG
KSWHSARNDSWTRAMDPAPARAQPAMHTVNGYVNRSLPGLIGCHKKSVYWHVIG
MGTSPEVHSIFLEGHTFLVRHHRQASLEISPLTFLTAQTFLMDLGQFLLFCHISSHHHG
GMEAHVRVESCAEEPQLRRKADEEEDYDDNLYDSDMDVVRLDGDDVSPFIQIRSVA
KKHPKTWVHYIAAEEEDWDYAPLVLAPDDRSYKSQYLNNGPQRIGRKYKKVRFMA
YTDETFKTREAIQHESGILGPLLYGEVGDTLLIIFKNQASRPYNIYPHGITDVRPLYSR
RLPKGVKHLKDFPILPGEIFKYKWTVTVEDGPTKSDPRCLTRYYSSFVNMERDLASG
LIGPLLICYKESVDQRGNQIMSDKRNVILFSVFDENRSWYLTENIQRFLPNPAGVQLE
DPEFQASNIMHSINGYVFDSLQLSVCLHEVAYWYILSIGAQTDFLSVFFSGYTFKHK
MVYEDTLTLFPFSGETVFMSMENPGLWILGCHNSDFRNRGMTALLKVSSCDKNTGD
YYEDSYEDISAYLLSKNNAIEPRSFAQNSRPPSASAPKPPVLRRHQRDISLPTFQPEED
KMDYDDIFSTETKGEDFDIYGEDENQDPRSFQKKTRHYFIAAVERLWDYGMSSSPH
VLRNRAQSGSVPQFKKVVFQEFTDGSFTQPLYRGELNEHLGLLGPYIRAEVEDNIMV
TFRNQASRPYSFYSSLISYEEDQRQGAEPRKNFVKPNETKTYFWKVQHHMAPTKDE
FDCKAWAYFSDVDLEKDVHSGLIGPLLVCHTNTLNPAHGRQVTVQEFALFFTIFDET
KSWYFTENMERNCRAPCNIQMEDPTFKENYRFHAINGYIMDTLPGLVMAQDQRIR
WYLLSMGSNENIHSIHFSGHVFSVRKKEEYKMAVYNLYPGVFETVEMLPSKVGIWR
IECLIGEHLQAGMSTTFLVYSKKCQTPLGMASGHIRDFQITASGQYGQWAPKLARLH
YSGSINAWSTKEPFSWIKVDLLAPMIIHGIKTQGARQKFSSLYISQFIIMYSLDGKKW
QTYRGNSTGTLMVFFGNVDSSGIKHNIFNPPIIARYIRLHPTHYSIRSTLRMELMGCDL
NSCSMPLGMESKAISDAQITASSYFTNMFATWSPSKARLHLQGRSNAWRPQVNNPK
EWLQVDFQKTMKVTGVTTQGVKSLLTSMYVKEFLISSSQDGHQWTLFFQNGKVKV
FQGNQDSFTPVVNSLDPPLLTRYLRIHPQSWVHQIALRMEVLGCEAQDLY
ETX-A3-FV
(SEQ ID NO: 17)
MQLELSTCVFLCLLPLGFSAIRRYYLGAVELSWDYRQSELLRELHVDTRFP
ATAPGALPLGPSVLYKKTVFVEFTDQLFSVARPRPPWMGLLGPTIQAEVYDTVVVTL
KNMASHPVSLHAVGVSFWKSSEGAEYEDHTSQREKEDDKVLPGKSQTYVWQVLKE
NGPTASDPPCLTYSYLSHVDLVKDLNSGLIGALLVCREGSLTRERTQNLHEFVLLFA
VFDEGKSWHSARNDSWTRAMDPAPARAQPAMHTVNGYVNRSLPGLIGCHKKSVY
WHVIGMGTSPEVHSIFLEGHTFLVRHHRQASLEISPLTFLTAQTFLMDLGQFLLFCHIS
SHHHGGMEAHVRVESCAEEPQLRRKADEEEDYDDNLYDSDMDVVRLDGDDVSPFI
QIRSVAKKHPKTWVHYIAAEEEDWDYAPLVLAPDDRSYKSQYLNNGPQRIGRKYK
KVRFMAYTDETFKTREAIQHESGILGPLLYGEVGDTLLIIFKNQASRPYNIYPHGITDV
RPLYSRRLPKGVKHLKDFPILPGEIFKYKWTVTVEDGPTKSDPRCLTRYYSSFVNME
RDLASGLIGPLLICYKESVDQRGNQIMSDKRNVILFSVFDENRSWYLTENIQRFLPNP
AGVQLEDPEFQASNIMHSINGYVFDSLQLSVCLHEVAYWYILSIGAQTDFLSVFFSGY
TFKHKMVYEDTLTLFPFSGETVFMSMENPGLWILGCHNSDFRNRGMTALLKVSSCD
KNTGDYYEDSYEDISAYLLSKNNAIEPRSFAQNSRPPSASAPKPPVLRRHQRDISLPTF
QPEEDKMDYDDIFSTETKGEDFDIYGEDENQDPRSFQKKTRHYFIAAVERLWDYGM
SESPHVLRNRAQSGSVPQFKKVVFREFTDGSFTQPLYRGELNEHLGLLGPYIRAEVE
DNIMVTFKNQASRPYSFYSSLISYEEDQRQGAEPRKNFVQPNETKTYFWKVQHHMA
PTKDEFDCKAWAYFSDVDLEKDVHSGLIGPLLICHTNTLNPAHGRQVTVQEFALFFT
IFDETKSWYFTENMERNCRAPCNLQMEDPTFKENYRFHAINGYIMDTLPGLVMAQD
QRIRWYLLSMGSNENIHSIHFSGHVFTVRKKEEYKMAVYNLYPGVFETVEMLPSKA
GIWRVECLIGEHLHAGMSTLFLVYSNKCQTPLGMASGHIRDFQITASGQYGQWAPK
LARLHYSGSINAWSTKEPFSWIKVDLLAPMIIHGIKTQGARQKFSSLYISQFIIMYSLD
GKKWQTYRGNSTGTLMVFFGNVDSSGIKHNIFNPPIIARYIRLHPTHYSIRSTLRMEL
MGCDLNSCSMPLGMESKAISDAQITASSYFTNMFATWSPSKARLHLQGRSNAWRPQ
VNNPKEWLQVDFQKTMKVTGVTTQGVKSLLTSMYVKEFLISSSQDGHQWTLFFQN
GKVKVFQGNQDSFTPVVNSLDPPLLTRYLRIHPQSWVHQIALRMEVLGCEAQDLY
ETX-A3-A11
(SEQ ID NO: 18)
MQLELSTCVFLCLLPLGFSAIRRYYLGAVELSWDYRQSELLRELHVDTRFP
ATAPGALPLGPSVLYKKTVFVEFTDQLFSVARPRPPWMGLLGPTIQAEVYDTVVVTL
KNMASHPVSLHAVGVSFWKSSEGAEYEDHTSQREKEDDKVLPGKSQTYVWQVLKE
NGPTASDPPCLTYSYLSHVDLVKDLNSGLIGALLVCREGSLTRERTQNLHEFVLLFA
VFDEGKSWHSARNDSWTRAMDPAPARAQPAMHTVNGYVNRSLPGLIGCHKKSVY
WHVIGMGTSPEVHSIFLEGHTFLVRHHRQASLEISPLTFLTAQTFLMDLGQFLLFCHIS
SHHHGGMEAHVRVESCAEEPQLRRKADEEEDYDDNLYDSDMDVVRLDGDDVSPFI
QIRSVAKKHPKTWVHYIAAEEEDWDYAPLVLAPDDRSYKSQYLNNGPQRIGRKYK
KVRFMAYTDETFKTREAIQHESGILGPLLYGEVGDTLLIIFKNQASRPYNIYPHGITDV
RPLYSRRLPKGVKHLKDFPILPGEIFKYKWTVTVEDGPTKSDPRCLTRYYSSFVNME
RDLASGLIGPLLICYKESVDQRGNQIMSDKRNVILFSVFDENRSWYLTENIQRFLPNP
AGVQLEDPEFQASNIMHSINGYVFDSLQLSVCLHEVAYWYILSIGAQTDFLSVFFSGY
TFKHKMVYEDTLTLFPFSGETVFMSMENPGLWILGCHNSDFRNRGMTALLKVSSCD
KNTGDYYEDSYEDISAYLLSKNNAIEPRSFAQNSRPPSASAPKPPVLRRHQRDISLPTF
QPEEDKMDYDDIFSTETKGEDFDIYGEDENQDPRSFQKKTRHYFIAAVERLWDYGM
SESPHVLRNRAQNGEVPQFKKVVFREFTDGSFTQPLYRGELNEHLGLLGPYIRAEVE
DNIMVTFKNQASRPYSFYSSLISYEEDQRQGAEPRKNFVQPNETKTYFWKVQHHMA
PTKDEFDCKAWAYFSDVDLEKDVHSGLIGPLLICRTNTLNPAHGRQVTVQEFALFFT
IFDETKSWYFTENMERNCRAPCNLQMEDPTFKENYRFHAINGYIMDTLPGLVMAQD
QRIRWYLLSMGSNENIHSIHFSGHVFTVRKKEEYKMAVYNLYPGVFETVEMLPSKA
GIWRVECLIGEHLHAGMSTTFLVYSNKCQTPLGMASGHIRDFQITASGQYGQWAPK
LARLHYSGSINAWSTKEPFSWIKVDLLAPMIIHGIKTQGARQKFSSLYISQFIIMYSLD
GKKWQTYRGNSTGTLMVFFGNVDSSGIKHNIFNPPIIARYIRLHPTHYSIRSTLRMEL
MGCDLNSCSMPLGMESKAISDAQITASSYFTNMFATWSPSKARLHLQGRSNAWRPQ
VNNPKEWLQVDFQKTMKVTGVTTQGVKSLLTSMYVKEFLISSSQDGHQWTLFFQN
GKVKVFQGNQDSFTPVVNSLDPPLLTRYLRIHPQSWVHQIALRMEVLGCEAQDLY
ETX-hSP, which contains the porcine ap-A3 domain and human
signal peptide, A2, C2 and C3 domains (FIG. 4), was prepared as
described previously (Barrow et al. (2000) Blood 95: 557-561).
(SEQ ID NO: 19)
MQIELSTCFFLCLLRFCFSAIRRYYLGAVELSWDYRQSELLRELHVDTRFPATAPGAL
PLGPSVLYKKTVFVEFTDQLFSVARPRPPWMGLLGPTIQAEVYDTVVVTLKNMASH
PVSLHAVGVSFWKSSEGAEYEDHTSQREKEDDKVLPGKSQTYVWQVLKENGPTAS
DPPCLTYSYLSHVDLVKDLNSGLIGALLVCREGSLTRERTQNLHEFVLLFAVFDEGK
SWHSARNDSWTRAMDPAPARAQPAMHTVNGYVNRSLPGLIGCHKKSVYWHVIGM
GTSPEVHSIFLEGHTFLVRHHRQASLEISPLTFLTAQTFLMDLGQFLLFCHISSHHHGG
MEAHVRVESCAEEPQLRRKADEEEDYDDNLYDSDMDVVRLDGDDVSPFIQIRSVAK
KHPKTWVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGITDVRPLYSRR
LPKGVKHLKDFPILPGEIFKYKWTVTVEDGPTKSDPRCLTRYYSSFVNMERDLASGL
IGPLLICYKESVDQRGNQIMSDKRNVILFSVFDENRSWYLTENIQRFLPNPAGVQLED
PEFQASNIMHSINGYVFDSLQLSVCLHEVAYWYILSIGAQTDFLSVFFSGYTFKHKM
VYEDTLTLFPFSGETVFMSMENPGLWILGCHNSDFRNRGMTALLKVSSCDKNTGDY
YEDSYEDISAYLLSKNNAIEPRSFAQNSRPPSASAPKPPVLRRHQRDISLPTFQPEEDK
MDYDDIFSTETKGEDFDIYGEDENQDPRSFQKRTRHYFIAAVEQLWDYGMSESPRA
LRNRAQNGEVPRFKKVVFREFADGSFTQPSYRGELNKHLGLLGPYIRAEVEDNIMVT
FKNQASRPYSFYSSLISYPDDQEQGAEPRHNFVQPNETRTYFWKVQHHMAPTEDEF
DCKAWAYFSDVDLEKDVHSGLIGPLLICRANTLNAAHGRQVTVQEFALFFTIFDETK
SWYFTENVERNCRAPCHLQMEDPTLKENYRFHAINGYVMDTLPGLVMAQNQRIRW
YLLSMGSNENIHSIHFSGHVFSVRKKEEYKMAVYNLYPGVFETVEMLPSKVGIWRIE
CLIGEHLQAGMSTTFLVYSKKCQTPLGMASGHIRDFQITASGQYGQWAPKLARLHY
SGSINAWSTKEPFSWIKVDLLAPMIIHGIKTQGARQKFSSLYISQFIIMYSLDGKKWQT
YRGNSTGTLMVFFGNVDSSGIKHNIFNPPIIARYIRLHPTHYSIRSTLRMELMGCDLNS
CSMPLGMESKAISDAQITASSYFTNMFATWSPSKARLHLQGRSNAWRPQVNNPKEW
LQVDFQKTMKVTGVTTQGVKSLLTSMYVKEFLISSSQDGHQWTLFFQNGKVKVFQ
GNQDSFTPVVNSLDPPLLTRYLRIHPQSWVHQIALRMEVLGCEAQDLY
ETX-SQ, which contains the porcine signal peptide, A1, ap-A3
domains, the porcine-derived linker sequence SFAQNSRPPSASAPKPPVLRR
HQR (SEQ ID NO: 11) and the human A2, C2 and C3 domains (FIG. 4),
was prepared as described previously (Barrow et al. (2000) Blood
95: 557-561).
(SEQ ID NO: 20)
MQLELSTCVFLCLLPLGFSAIRRYYLGAVELSWDYRQSELLRELHVDTRFP
ATAPGALPLGPSVLYKKTVFVEFTDQLFSVARPRPPWMGLLGPTIQAEVYDTVVVTL
KNMASHPVSLHAVGVSFWKSSEGAEYEDHTSQREKEDDKVLPGKSQTYVWQVLKE
NGPTASDPPCLTYSYLSHVDLVKDLNSGLIGALLVCREGSLTRERTQNLHEFVLLFA
VFDEGKSWHSARNDSWTRAMDPAPARAQPAMHTVNGYVNRSLPGLIGCHKKSVY
WHVIGMGTSPEVHSIFLEGHTFLVRHHRQASLEISPLTFLTAQTFLMDLGQFLLFCHIS
SHHHGGMEAHVRVESCAEEPQLRRKADEEEDYDDNLYDSDMDVVRLDGDDVSPFI
QIRSVAKKHPKTWVHYIAAEEEDWDYAPLVLAPDDRSYKSQYLNNGPQRIGRKYK
KVRFMAYTDETFKTREAIQHESGILGPLLYGEVGDTLLIIFKNQASRPYNIYPHGITDV
RPLYSRRLPKGVKHLKDFPILPGEIFKYKWTVTVEDGPTKSDPRCLTRYYSSFVNME
RDLASGLIGPLLICYKESVDQRGNQIMSDKRNVILFSVFDENRSWYLTENIQRFLPNP
AGVQLEDPEFQASNIMHSINGYVFDSLQLSVCLHEVAYWYILSIGAQTDFLSVFFSGY
TFKHKMVYEDTLTLFPFSGETVFMSMENPGLWILGCHNSDFRNRGMTALLKVSSCD
KNTGDYYEDSYEDISAYLLSKNNAIEPRSFSQNPPVLKRHQRDISLPTFQPEEDKMDY
DDIFSTETKGEDFDIYGEDENQDPRSFQKRTRHYFIAAVEQLWDYGMSESPRALRNR
AQNGEVPRFKKVVFREFADGSFTQPSYRGELNKHLGLLGPYIRAEVEDNIMVTFKN
QASRPYSFYSSLISYPDDQEQGAEPRHNFVQPNETRTYFWKVQHHMAPTEDEFDCK
AWAYFSDVDLEKDVHSGLIGPLLICRANTLNAAHGRQVTVQEFALFFTIFDETKSWY
FTENVERNCRAPCHLQMEDPTLKENYRFHAINGYVMDTLPGLVMAQNQRIRWYLL
SMGSNENIHSIHFSGHVFSVRKKEEYKMAVYNLYPGVFETVEMLPSKVGIWRIECLI
GEHLQAGMSTTFLVYSKKCQTPLGMASGHIRDFQITASGQYGQWAPKLARLHYSGS
INAWSTKEPFSWIKVDLLAPMIIHGIKTQGARQKFSSLYISQFIIMYSLDGKKWQTYR
GNSTGTLMVFFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNNPKEWL
QVDFQKTMKVTGVTTQGVKSLLTSMYVKEFLISSSQDGHQWTLFFQNGKVKVFQG
NQDSFTPVVNSLDPPLLTRYLRIHPQSWVHQIALRMEVLG
CEAQDLY
ETX-hSP-SQ which contains the porcine A1 domain, A2, and ap-A3,
and the human signal peptide, A2, C2 and C3 domains and the human
SFSQNPPVLKRHQR (SEQ ID NO: 11) linker sequence (FIG. 4), was
prepared by SOE mutagenesis.
(SEQ ID NO: 21)
MQIELSTCFFLCLLRFCFSAIRRYYLGAVELSWDYRQSELLRELHVDTRFP
ATAPGALPLGPSVLYKKTVFVEFTDQLFSVARPRPPWMGLLGPTIQAEVYDTVVVTL
KNMASHPVSLHAVGVSFWKSSEGAEYEDHTSQREKEDDKVLPGKSQTYVWQVLKE
NGPTASDPPCLTYSYLSHVDLVKDLNSGLIGALLVCREGSLTRERTQNLHEFVLLFA
VFDEGKSWHSARNDSWTRAMDPAPARAQPAMHTVNGYVNRSLPGLIGCHKKSVY
WHVIGMGTSPEVHSIFLEGHTFLVRHHRQASLEISPLTFLTAQTFLMDLGQFLLFCHIS
SHHHGGMEAHVRVESCAEEPQLRRKADEEEDYDDNLYDSDMDVVRLDGDDVSPFI
QIRSVAKKHPKTWVHYIAAEEEDWDYAPLVLAPDDRSYKSQYLNNGPQRIGRKYK
KVRFMAYTDETFKTREAIQHESGILGPLLYGEVGDTLLIIFKNQASRPYNIYPHGITDV
RPLYSRRLPKGVKHLKDFPILPGEIFKYKWTVTVEDGPTKSDPRCLTRYYSSFVNME
RDLASGLIGPLLICYKESVDQRGNQIMSDKRNVILFSVFDENRSWYLTENIQRFLPNP
AGVQLEDPEFQASNIMHSINGYVFDSLQLSVCLHEVAYWYILSIGAQTDFLSVFFSGY

TFKHKMVYEDTLTLFPFSGETVFMSMENPGLWILGCHNSDFRNRGMTALLKVSSCD
KNTGDYYEDSYEDISAYLLSKNNAIEPRSFSQNPPVLKRHQRDISLPTFQPEEDKMDY
DDIFSTETKGEDFDIYGEDENQDPRSFQKRTRHYFIAAVEQLWDYGMSESPRALRNR
AQNGEVPRFKKVVFREFADGSFTQPSYRGELNKHLGLLGPYIRAEVEDNIMVTFKN
QASRPYSFYSSLISYPDDQEQGAEPRHNFVQPNETRTYFWKVQHHMAPTEDEFDCK
AWAYFSDVDLEKDVHSGLIGPLLICRANTLNAAHGRQVTVQEFALFFTIFDETKSWY
FTENVERNCRAPCHLQMEDPTLKENYRFHAINGYVMDTLPGLVMAQNQRIRWYLL
SMGSNENIHSIHFSGHVFSVRKKEEYKMAVYNLYPGVFETVEMLPSKVGIWRIECLI
GEHLQAGMSTTFLVYSKKCQTPLGMASGHIRDFQITASGQYGQWAPKLARLHYSGS
INAWSTKEPFSWIKVDLLAPMIIHGIKTQGARQKFSSLYISQFIIMYSLDGKKWQTYR
GNSTGTLMVFFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNNPKEWL
QVDFQKTMKVTGVTTQGVKSLLTSMYVKEFLISSSQDGHQWTLFFQNGKVKVFQG
NQDSFTPVVNSLDPPLLTRYLRIHPQSWVHQIALRMEVLGCEAQDLY
ETX-s7-8 (A1 domain of HSQ): The A1 domain of HSQ with regions from
ET3 substituted in (48 mutations).
(SEQ ID NO: 22)
ATRRYYLGAVELSWDYMQSDLGELPVDARFPPRVPKSFPFNTSVVYKKT
LFVEFTDHLFNIAKPRPPWMGLLGPTIQAEVYDTVVITLKNMASHPVSLHAVGVSY
WKASEGAEYDDQTSQREKEDDKVFPGGSHTYVWQVLKENGPMASDPLCLTYSYLS
HVDLVKDLNSGLIGALLVCREGSLAKEKTQTLHKFILLFAVFDEGKSWHSETKNSL
MQDRDAASARAWPKMHTVNGYVNRSLPGLIGCHRKSVYWHVIGMGTTPEVHSIFL
EGHTFLVRNHRQASLEISPLTFLTAQTFLMDLGQFLLFCHISSHHHGGMEAHVRVES
CAEEPQLRRKADEEEDYDDNLYDSDMDVVRLDGDDVSPFIQIR
ETX-A1-FV (A1 domain of HSQ): A1 domain of HSQ with residues conserved in
ET3 and Human Factor V but no HSQ, substituted in to HSQ (10 mutations).
(SEQ ID NO: 23)
ATRRYYLGAVELSWDYRQSDLGELPVDARFPPRVPKSFPFNTSVVYKKTV
FVEFTDHLFNIAKPRPPWMGLLGPTIQAEVYDTVVVTLKNMASHPVSLHAVGVSYW
KASEGAEYDDHTSQREKEDDKVFPGGSHTYVWQVLKENGPTASDPPCLTYSYLSHV
DLVKDLNSGLIGALLVCREGSLTKEKTQTLHKFVLLFAVFDEGKSWHSETKNSLMQ
DRDAASARAWPKMHTVNGYVNRSLPGLIGCHRKSVYWHVIGMGTTPEVHSIFLEG
HTFLVRHHRQASLEISPITFLTAQTLLMDLGQFLLFCHISSHQHDGMEAYVKVDSCPE
EPQLRMKANEEAEDYDDDLTDSEMDVVRFDDDNSPSFIQIR
ETX-A1 All (A1 Domain): A1 domain of HSQ with all residues conserved in
ET3, hcP and/or human Factor V but not HSQ substituted into HSQ (15
mutations).
(SEQ ID NO: 24)
ATRRYYLGAVELSWDYRQSDLGELPVDTRFPPRVPKSFPFNTSVLYKKTV
FVEFTDHLFNIAKPRPPWMGLLGPTIQAEVYDTVVVTLKNMASHPVSLHAVGVSYW
KASEGAEYDDHTSQREKEDDKVFPGGSHTYVWQVLKENGPTASDPPCLTYSYLSHV
DLVKDLNSGLIGALLVCREGSLTKEKTQTLHEFVLLFAVFDEGKSWHSETKDSLMQ
DRDAASARAWPKMHTVNGYVNRSLPGLIGCHRKSVYWHVIGMGTTPEVHSIFLEG
HTFLVRHHRQASLEISPITFLTAQTLLMDLGQFLLFCHISSHQHDGMEAYVKVDSCPE
EPQLRMKANEEAEDYDDNLTDSEMDVVRFDDDNSPSFIQIR
ETX-A3-FV: A3 domain of HSQ with residues conserved in ET3 and hfV
but not HSQ substituted into HSQ (7 substitutions).
(SEQ ID NO: 25)
SFQKKTRHYFIAAVERLWDYGMSESPHVLRNRAQSGSVPQFKKVVFREFT
DGSFTQPLYRGELNEHLGLLGPYIRAEVEDNIMVTFKNQASRPYSFYSSLISYEEDQR
QGAEPRKNFVQPNETKTYFWKVQHHMAPTKDEFDCKAWAYFSDVDLEKDVHSGLI
GPLLICHTNTLNPAHGRQVTVQEFALFFTIFDETKSWYFTENMERNCRAPCNLQMED
PTFKENYRFHAINGYIMDTLPGLVMAQDQRIRWYLLSMGSNENIHSIHFSGHVFTVR
KKEEYKMAVYNLYPGVFETVEMLPSKAGIWRVECLIGEHLHAGMSTLFLVYSN
ETX-A3-All: A3 domain of HSQ with all residues conserved in ET3, hcP
and/or hfV but not HSQ substituted into HSQ (11 substitutions).
(SEQ ID NO: 26)
SFQKKTRHYFIAAVERLWDYGMSESPHVLRNRAQNGEVPQFKKVVFREF
TDGSFTQPLYRGELNEHLGLLGPYIRAEVEDNIMVTFKNQASRPYSFYSSLISYEEDQ
RQGAEPRKNFVQPNETKTYFWKVQHHMAPTKDEFDCKAWAYFSDVDLEKDVHSG
LIGPLLICRTNTLNPAHGRQVTVQEFALFFTIFDETKSWYFTENMERNCRAPCNLQM
EDPTFKENYRFHAINGYIMDTLPGLVMAQDQRIRWYLLSMGSNENIHSIHFSGHVFT
VRKKEEYKMAVYNLYPGVFETVEMLPSKAGIWRVECLIGEHLHAGMSTTFLVYSN
ETX-A3-CU: HSQ A3 domain with sequence of ET3 surrounding the
A3 copper binding region substituted into the HSQ A3 (7 substitutions).
(SEQ ID NO: 27)
SFQKKTRHYFIAAVERLWDYGMSSSPHVLRNRAQSGSVPQFKKVVFQEFT
DGSFTQPLYRGELNEHLGLLGPYIRAEVEDNIMVTFRNQASRPYSFYSSLISYEEDQR
QGAEPRKNFVKPNETKTYFWKVQHHMAPTKDEFDCKAWAYFSDVDLEKDVHSGLI
GPLLVCHTNTLNPAHGRQVTVQEFALFFTIFDETKSWYFTENMERNCRAPCNIQME
DPTFKENYRFHAINGYIMDTLPGLVMAQDQRIRWYLLSMGSNENIHSIHFSGHVFSV
RKKEEYKMAVYNLYPGVFETVEMLPSKVGIWRIECLIGEHLQAGMSTTFLVYSK

Sequences produced by SOE mutagenesis were confirmed by dideoxy DNA sequencing.

Transient Expression of Factor VIII from COS-7 Cells

COS-7 cells were grown to 70-80% confluence in 2 cm² wells containing 1 ml DMEM:F12 supplemented with 10% FBS, 100 units/ml penicillin and 100 μg/ml streptomycin. Cells were transfected with a 2000:1 mass ratio of factor VIII plasmid:luciferase plasmid DNA using Lipofectamine 2000. Twenty-four hours after transfection the cells were rinsed twice with 1 ml of PBS and 0.5 ml of serum-free AIM V medium was added to each well. Cells were cultured 24 hr before the conditioned media was harvested and factor VIII activity was measured as described below.

Stable Expression of Factor VIII from Baby Hamster Kidney-Derived (BHK-M) Cells

BHK-M cells were transfected using Lipofectin along with an ReNeo plasmid containing factor VIII cDNA and cultured in the presence of DMEM:F12 containing 10% FBS, 100 units/ml penicillin, 100 μg/ml streptomycin and 500 μg/ml geneticin for 10 days. The ReNeo vector contains the neomycin phosphotransferase gene for resistance to the antibiotic geneticin. Twenty-four to 72 geneticin resistant clones were screened for factor VIII production. The clone from each cDNA construct that displayed the highest level of factor VIII activity was transferred into a 75 cm² flask, grown to 90-95% confluence and then switched to 25 ml serum-free AIM V media. After 24 hr, the conditioned media was replaced with 25 ml fresh serum-free media AIM V and cultured for an additional 24 hr. Harvested media from each time point was assayed for factor VIII activity as described below.

### Factor VIII Assay

Factor VIII activity was measured by one-stage coagulation assay using a ST art Coagulation Instrument (Diagnostica Stago, Asnieres, France). Five μl of sample or standard was added to 50 μl of factor VIII-deficient plasma, followed by addition of 50 μl aPTT reagent and incubation for 3 min at 37° C. Fifty microliters of 20 mM CaCl₂ was added to initiate the reaction, and the time required to develop a fibrin clot was measured viscometrically. Standard curves were generated using several dilutions of pooled normal human plasma and subjected to linear regression analysis of the clotting time versus the logarithm of the reciprocal plasma dilution. For determination of factor VIII activity, samples were diluted in HEPES buffered saline to a concentration within the range of the standard curve.

### Results

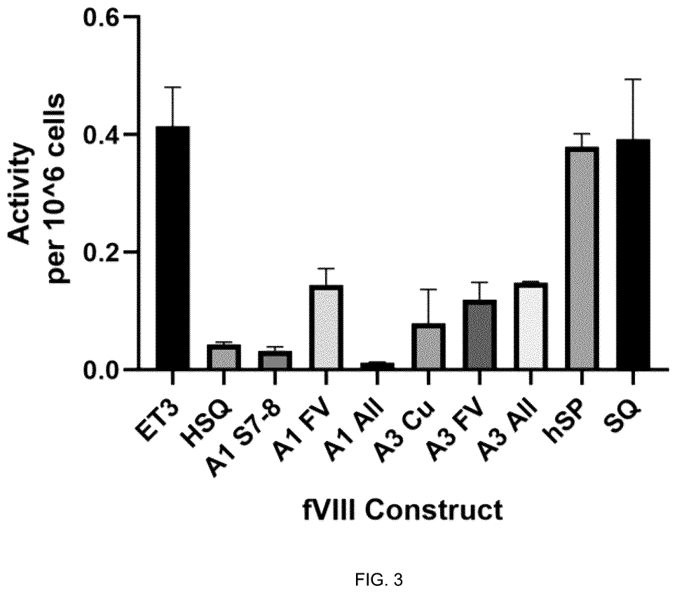

To identify constructs that exhibit high-level expression, variant factor VIII molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) were constructed and their expression levels in COS-7 and BHK-M cells were measured. After COS-7 cell transfection, the expression plasmid is not integrated into genomic DNA, but is present transiently as an episomal DNA. Expression levels from COS-7 cells represent an average of the cell population. FIG. 3 shows the results of COS-7 wells transfected in quadruplicate. There is a significant increase in expression of ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) compared to HSQ. In contrast, expression of A1 S7-8 and A1-All were not increased compared to HSQ.

Average expression levels for factor VIII-producing clones were significantly higher for the ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) constructs, compared to HSQ (SEQ ID NO: 28) (data not shown).

### DISCUSSION

Recombinant B domain-deleted porcine factor VIII is expressed at levels up to 14-fold greater than recombinant human factor VIII (Doering et al. (2002) J. Biol. Chem. 277: 38345-38349). The levels are substantially greater than in previously published reports of factor VIII expression (Table II). The mechanism for the high expression phenomenon has not been established. However, high-level expression is due to a difference between human and porcine B domain-deleted factor VIII in translated sequence because the P/OL and HSQ expression cassettes do not contain endogenous factor VIII 5′-UTR sequence, while both possess the first 749 nt (of 1805 nt) of the human factor VIII 3′UTR. Furthermore, the effect occurs at the post-transcriptional level, because there is no difference in P/OL and HSQ mRNA levels in BHK-M cells (Doering et al. (2002) J. Biol. Chem. 277:38345-38349).

TABLE II
Previous Reports of FACTOR VIII Expression.

| FACTOR VIII | FVIII | | | | Cell | |
|---|---|---|---|---|---|---|
| Construct | Level | Assay | Serum | vWf | Line | Reference |
| Human, full length | 0.07^{a} | Coatest | + | − | BHK | Wood et al. (1984) |
| | | | | | | Nature 312:330-337 |
| Human, full length | 0.16^{a} | Coatest | + | − | COS | Toole et al. |
| | 0.33^{a} | Coagulation | | | | (1986) Proc. Natl. |
| | | | | | | Acad. Sci. U.S.A. |
| | | | | | | 83:5939-5942 |
| Human, B domain- | 0.34^{a} | Coatest | − | − | CHO^{c} | Kaufman et al. |
| deleted | | | | | | (1988) J.Biol.Chem. |
| | | | | | | 263:6352-6362 |
| Human, full length | 1.4^{b} | Coatest | − | + | CHO | Kaufman et al. |
| | | | | | | (1989) Mol.Cell |
| | | | | | | Biol. 9:1233-1242 |
| Human, B domain- | 5^{a} | Coatest | − | + | CHO | Pittman et al. (1993) |
| deleted | | | | | | Blood 81:2925-2935 |
| Human, B domain- | 1.5^{a} | Coatest | − | − | CHO | Lind et al (1995) |
| deleted | | | | | | Eur. J. Biochem. |
| | | | | | | 232:19-27 |
| Human, B domain- | 2.5^{b} | Coagulation | + | − | CHO | Plantier et al. (2001) |
| deleted | | | | | | Thromb. Haemost. |
| | | | | | | 86:596-603 |
| Human, B domain- | 3.1^{a} | Coagulation | − | − | BHK | Deering et al. (2002) |
| deleted | 10^{b} | | | | | J.Biol. Chem. 277, |
| | | | | | | 38345-38349 |
| Porcine, B domain- | 41^{a} | Coagulation | − | − | BHK | Deering et al. (2002) |
| deleted | 140^{b} | | | | | J.Biol. Chem. 277, |
| | | | | | | 38345-38349 |

^{a}units/milliliter/24 hours
^{b}units/106 cells/24 hours
^{c}Chinese hamster ovary

### Example 3

Variants of the factor VIII_{SEP} sequences may be generated. For example, the ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) factor VIII_{SEP} may be generated.

Two major human factor VIII epitopes that are recognized by inhibitory antibodies have been identified: in the A2 domain in a segment bound by residues 484-508 (Healey et al. (1995) J. Biol. Chem. 270:14505-14509) and in the C2 domain in a segment bounded by residues 2181-2252 (Healey et al. (1998) Blood 92:3701-3709 and Barrow et al. (2001) Blood 97:169-174, all of which are herein incorporated by reference). The sequence numbering refers to the full-length, mature human factor VIII according to standard convention (Vehar et al. (1984) Nature 312:337-342). Antibodies also have been identified that recognize the light chain activation peptide, ap, (Barrow et al. (2000) Blood 95:557-561) and the A3 domain in a region bounded by residues 1804-1819 (Zhong et al. (1998) Blood 92:136-142), but they are less common (Prescott et al. (1997) Blood 89:3663-3671). Other epitopes occasionally have been identified, but they are considered unusual.

TABLE III
Sequence ID Listing

| SEQ ID | | | |
|---|---|---|---|
| NO. | Type | Species | Description |
| 1 | NT | Sus scrofa | Factor VIII |
| 2 | AA | Sus scrofa | Factor VIII |
| 3 | NT | Sus scrofa | Factor VIII - B-domain deleted |
| | | | (retains first 12 and last 12 |
| | | | amino acids of B-domain) |
| 4 | AA | Sus scrofa | Factor VIII - B domain deleted |
| | | | (retains first 12 and last 12 |
| | | | amino acids of B-domain) |
| 5 | NT | Homo sapiens | Factor VIII with 5′ and 3′ UTR |
| | | | sequences |
| 6 | AA | Homo sapiens | Factor VIII |
| 7 | NT | Homo sapiens | Factor VIII cDNA |
| 8 | AA | Mus musculus | Factor VIII |
| 9 | AA | Homo sapiens | Linker sequence between A2 and |
| | | | ap domains |
| 10 | AA | Homo sapiens | Recognition sequence for PACE/furin |
| 11 | AA | Sus scrofa | Linker sequence between A2 and |
| | | | ap domains |
| 12 | NT | Homo sapiens | Factor VIII - B-domain deleted |
| 13 | AA | Homo sapiens | Factor VIII - B-domain deleted |
| 14 | AA | Artificial | ET-3 Factor VIII |
| 15 | AA | Artificial | ETX-A1-fV |
| 16 | AA | Artificial | ETX-A3-Cu |
| 17 | AA | Artificial | ETX-A3-fV |
| 18 | AA | Artificial | ETX-A3-all |
| 19 | AA | Artificial | ETX-hSP |
| 20 | AA | Artificial | ETX-SQ |
| 21 | AA | Artificial | ETX-hSP-SQ |
| 22 | AA | Artificial | ETx s7-8 (Al domain of HSQ) |
| 23 | AA | Artificial | ETX A1-fV (A1 domain of HSQ) |
| 24 | AA | Artificial | ETX A1 All (A1 domain of HSQ) |
| 25 | AA | Artificial | ETX A3 fV (A3 domain of HSQ) |
| 26 | AA | Artificial | ETX A3 all (A3 domain of HSQ) |
| 27 | AA | Artifical | ETX A3 Cu (A3 domain of HSQ) |
| 28 | AA | Artificial | HSQ |

Therapeutics

The nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) encoding a FVIII protein, or variant thereof, can be included in a vector (such as a AAV vector) for expression in a cell or a subject.

The nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) encoding a FVIII protein are useful in production of vectors (such as rAAV vectors), and are also useful in antisense delivery vectors, gene therapy vectors, or vaccine vectors. In certain embodiments, the disclosure provides for gene delivery vectors, and host cells which contain the nucleic acid sequences disclosed herein. In some embodiments, the selected vector may be delivered to a subject by any suitable method, including intravenous injection, ex-vivo transduction, transfection, electroporation, liposome delivery, membrane fusion techniques, high velocity DNA-coated pellets, viral infection, or protoplast fusion, to introduce a transgene into the subject.

In certain embodiments, the disclosure relates to virus particle, e.g., capsids, containing the nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) encoding a FVIII protein disclosed herein. The virus particles, capsids, and recombinant vectors are useful in delivery of the nucleic acid sequences encoding the FVIII proteins to a target cell. The nucleic acids may be readily utilized in a variety of vector systems, capsids, and host cells. In certain embodiments, the nucleic acids are in vectors contained within a capsid comprising cap proteins, including AAV capsid proteins vp1, vp2, vp3 and hypervariable regions.

In certain embodiments, nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) may be a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon.

In certain embodiments, a vector may be a lentivirus based (containing lentiviral genes or sequences) vector, e.g., having nucleic acid sequences derived from VSVG or GP64 pseudotypes or both. In certain embodiments, the nucleic acid sequences derived from VSVG or GP64 pseudotypes may be at least one or two or more genes or gene fragments of more than 1000, 500, 400, 300, 200, 100, 50, or 25 continuous nucleotides or nucleotides sequences with greater than 50, 60, 70, 80, 90, 95 or 99% identity to the gene or fragment.

In some embodiments, the nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) disclosed herein are useful in production of AAV vectors. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008).

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008).

AAV vectors typically contain a transgene expression cassette between the ITRs that replaces the rep and cap genes. Vector particles are produced by the co-transfection of cells with a plasmid containing the vector genome and a packaging/helper construct that expresses the rep and cap proteins in trans. During infection, AAV vector genomes enter the cell nucleus and can persist in multiple molecular states. One common outcome is the conversion of the AAV genome to a double-stranded circular episome by second-strand synthesis or complementary strand pairing.

In the context of AAV vectors, the disclosed vectors typically have a recombinant genome comprising the following structure:
(5′AAV ITR)-(promoter)-(transgene)-(3′AAV ITR)

As discussed above, these recombinant AAV vectors contain a transgene expression cassette between the ITRs that replaces the rep and cap genes. Vector particles are produced, for example, by the co-transfection of cells with a plasmid containing the recombinant vector genome and a packaging/helper construct that expresses the rep and cap proteins in trans.

The transgene can be flanked by regulatory sequences such as a 5′ Kozak sequence and/or a 3′ polyadenylation signal.

The AAV ITRs, and other selected AAV components described herein, may be readily selected from among any AAV serotype, including, without limitation, AAV1, AAV2, AAV3, AAV4, AAV5, AAV6, AAV7, AAV8, AAV9 and function variants thereof. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

In some embodiments, the recombinant AAV vector genome can have a liver-specific promoter, such as any one of the HCB, HSh-HCB, 5′HSh-HCB, 3′HSh-HCB, ABP-HP1-God-TSS, HSh-SynO-TSS, or sHS-SynO-TSS promoters set forth in WO 2016/168728, which is incorporated by reference herein in its entirety.

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev 21(4):583-593, 2008).

Methods for producing rAAV suitable for gene therapy are known (see, for example, U.S. Patent Application Nos. 2012/0100606; 2012/0135515; 2011/0229971; and 2013/0072548; and Ghosh et al., Gene Ther 13(4):321-329, 2006), and can be utilized with the recombinant nucleic acid molecules and methods disclosed herein.

In some embodiments, the nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) disclosed herein are part of an expression cassette or transgene. See e.g., US Pat. App. Pub. 20150139953. The expression cassette is composed of a transgene and regulatory sequences, e.g., promotor and 5′ and 3′ AAV inverted terminal repeats (ITRs). In one desirable embodiment, the ITRs of AAV serotype 2 or 8 are used. However, ITRs from other suitable serotypes may be selected. An expression cassette is typically packaged into a capsid protein and delivered to a selected host cell.

In some embodiments, the disclosure provides for a method of generating a recombinant adeno-associated virus (AAV) having an AAV serotype capsid, or a portion thereof. Such a method involves culturing a host cell which contains a nucleic acid sequence encoding an adeno-associated virus (AAV) serotype capsid protein; a functional rep gene; an expression cassette composed of AAV inverted terminal repeats (ITRs) and a transgene; and sufficient helper functions to permit packaging of the expression cassette into the AAV capsid protein. See e.g., US Pat. App. Pub. 20150139953.

The components for culturing in the host cell to package an AAV expression cassette in an AAV capsid may be provided to the host cell in trans. Alternatively, any one or more of the components (e.g., expression cassette, rep sequences, cap sequences, and/or helper functions) may be provided by a stable host cell which has been engineered to contain one or more of the required components using methods known to those of skill in the art. Most suitably, such a stable host cell will contain the component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

In some embodiments, the disclosure relates to recombinant vectors comprising a nucleic acids encoding any of the above discussed recombinant amino acid molecules ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) in operable combination with transgene. The transgene is a nucleic acid sequence, heterologous to the vector sequences flanking the transgene, which encodes a novel FVIII protein as disclosed herein, and optionally one or more additional proteins of interest. The nucleic acid coding sequence is operatively linked to regulatory components in a manner which permits transgene transcription, translation, and/or expression in a host cell.

The expression cassette can be carried on any suitable vector, e.g., a plasmid, which is delivered to a host cell. The plasmids useful in this disclosure may be engineered such that they are suitable for replication and, optionally, integration in prokaryotic cells, mammalian cells, or both. These plasmids (or other vectors carrying the 5′ AAV ITR-heterologous molecule-3′ ITR) contain sequences permitting replication of the expression cassette in eukaryotes and/or prokaryotes and selection markers for these systems. Preferably, the molecule carrying the expression cassette is transfected into the cell, where it may exist transiently. Alternatively, the expression cassette (carrying the 5′ AAV ITR-heterologous molecule-3′ ITR) may be stably integrated into the genome of the host cell, either chromosomally or as an episome. In certain embodiments, the expression cassette may be present in multiple copies, optionally in head-to-head, head-to-tail, or tail-to-tail concatamers. Suitable transfection techniques are known and may readily be utilized to deliver the expression cassette to the host cell.

Generally, when delivering the vector comprising the expression cassette by transfection, the vector and the relative amounts of vector DNA to host cells may be adjusted, taking into consideration such factors as the selected vector, the delivery method and the host cells selected. In addition to the expression cassette, the host cell contains the sequences which drive expression of the AAV capsid protein in the host cell and rep sequences of the same serotype as the serotype of the AAV ITRs found in the expression cassette, or a cross-complementing serotype. Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell.

The packaging host cell also typically contains helper functions in order to package the rAAV of the disclosure. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). The desired helper functions, can be provided using any means that allows their expression in a cell.

Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously added factors, for example.

The AAV techniques can be adapted for use in these and other viral vector systems for in vitro, ex vivo or in vivo gene delivery. The in certain embodiments the disclosure contemplates the use of nucleic acids and vectors disclosed herein in a variety of rAAV and non-rAAV vector systems. Such vectors systems may include, e.g., lentiviruses, retroviruses, poxviruses, vaccinia viruses, and adenoviral systems, among others.

In some embodiments, it is contemplated that viral particles, nucleic acids and vectors disclosed herein are useful for a variety of purposes, including for delivery of therapeutic molecules for gene expression of therapeutic proteins.

Therapeutic proteins encoded by the nucleic acids (e.g., operably in combination with promoters) reported herein include those used for treatment of clotting disorders, including hemophilia A (e.g., using a fVIII protein as provided herein).

In some embodiments, a method of inducing blood clotting in a subject in need thereof is provided. The method comprises administering to the subject a therapeutically effective amount of a vector (such as an AAV vector, a lentiviral vector, or a retroviral vector) encoding a nucleic acid sequences encoding nucleic ETX-A1-FV (SEQ ID NO: 15), ETX-A3-Cu (SEQ ID NO: 16), ETX-A3-FV (SEQ ID NO: 17), ETX-A3-All (SEQ ID NO: 18), ETX-hSP (SEQ ID NO: 19), ETX-SQ (SEQ ID NO: 20), and ETX-hSP-SQ (SEQ ID NO: 21) FVIII proteins as described herein. In some embodiments, the subject is a subject with a clotting disorder, such as hemophilia A. In some embodiments, the clotting disorder is hemophilia A and the subject is administered a vector comprising a nucleic acid molecule encoding a protein with FVIII activity.

A treatment option for a patient diagnosed with hemophilia A is the exogenous administration of recombinant FVIII sometimes referred to as FVIII replacement therapy. In some embodiments, a patient with hemophilia A or of a recombinant fVIII protein as described herein. In some patients, these therapies can lead to the development of antibodies that bind to the administered clotting factor. Subsequently, the clotting factor-antibody bound conjugates, typically referred to as inhibitors, interfere with or retard the ability of the exogenous clotting factor to cause blood clotting. Inhibitory autoantibodies also sometimes occur spontaneously in a subject that is not genetically at risk of having a clotting disorder such as hemophilia, termed acquired hemophilia. Inhibitory antibodies assays are typically performed prior to exogenous clotting factor treatment in order to determine whether the anti-coagulant therapy will be effective.

A “Bethesda assay” has historically been used to quantitate the inhibitory strength the concentration of fVIII binding antibodies. In the assay, serial dilutions of plasma from a patient, e.g., prior to having surgery, are prepared and each dilution is mixed with an equal volume of normal plasma as a source of fVIII. After incubating for a couple hours, the activities of fVIII in each of the diluted mixtures are measured. Having antibody inhibitor concentrations that prevent fVIII clotting activity after multiple repeated dilutions indicates a heightened risk of uncontrolled bleeding. Patients with inhibitor titers after about ten dilutions are felt to be unlikely to respond to exogenous fVIII infusions to stop bleeding. A Bethesda titer is defined as the reciprocal of the dilution that results in 50% inhibition of FVIII activity present in normal human plasma. A Bethesda titer greater than 10 is considered the threshold of response to FVIII replacement therapy.

In certain embodiments, the disclosure relates to methods of inducing blood clotting comprising administering an effective amount of a viral particle or capsid comprising a vector comprising a nucleic acid encoding a blood clotting factor as disclosed herein to a subject in need thereof.

In certain embodiments, the subject is diagnosed with hemophilia A or acquired hemophilia or unlikely to respond to exogenous clotting factor infusions (e.g., based on a Bethesda assay result).

In some embodiments, this disclosure relates to methods of gene transfer for the treatment of hemophilia A using an adeno-associated viral (AAV) vector encoding human FVIII as the gene delivery vehicle. While several such AAV-based gene therapies for hemophilia A have entered into human clinical trials, they have been hampered by low expression of the therapeutic protein, clotting FVIII, after administration of the virus resulting on only partial correction of the disease. AAV vector toxicity limits the dose of the virus that may be safely administered. Typically, the vector provides efficacious expression of FVIII at viral doses below the threshold of toxicity.

In some embodiments, this disclosure relates to methods of gene transfer for the treatment of hemophilia A using a lentiviral vector encoding human FVIII as the gene delivery vehicle. Delivery of the lentiviral vector encoding the transgene can be, for example, by direct administration to the subject, or by ex vivo transduction and transplantation of hematopoietic stem and progenitor cells with the vector. Typically, the vector provides efficacious expression of FVIII at viral doses below the threshold of toxicity.

In some embodiments, recombinant virus particles, capsids, or vectors comprising nucleic acids disclosed herein can be delivered to liver via the hepatic artery, the portal vein, or intravenously to yield therapeutic levels of therapeutic proteins or clotting factors in the blood. The capsid or vector is preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, sesame oil, and water.

Optionally, the compositions of the disclosure may contain other pharmaceutically acceptable excipients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The recombinant virus particles, capsids, or vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intravenous, intramuscular, subcutaneous, intradermal, and other parental routes of administration. Routes of administration may be combined, if desired.

Dosages of the recombinant virus particles, capsids, or vectors will depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes virus vector.

Recombinant viral vectors of the disclosure provide an efficient gene transfer vehicle which can deliver a selected protein to a selected host cell in vivo or ex vivo even where the organism has neutralizing antibodies to the protein. In one embodiment, the vectors disclosed herein and the cells are mixed ex vivo; the infected cells are cultured using conventional methodologies; and the transduced cells are re-infused into the patient.

The present invention has been described above with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

What is claimed is:

1. An isolated modified factor VIII polypeptide comprising a nucleotide sequence having at least 95% sequence identity to the polynucleotide set forth as one of SEQ ID NO: 19, SEQ ID NO: 20, SEQ ID NO: 21, SEQ ID NO: 15, SEQ ID NO: 16, SEQ ID NO: 17, SEQ ID NO: 18, wherein said polypeptide is characterized by high-level expression when compared to a corresponding human factor VIII polypeptide expressed under the same conditions.

2. An isolated modified factor VIII polypeptide of claim 1, wherein said polypeptide comprises the amino acid sequence set forth as one of SEQ ID NO: 19, SEQ ID NO: 20, SEQ ID NO: 21, SEQ ID NO: 15, SEQ ID NO: 16, SEQ ID NO: 17, SEQ ID NO: 18.

3. A nucleic acid encoding the isolated modified factor VIII polypeptides of claim 1.

4. A pharmaceutical composition comprising the isolated modified factor VIII polypeptides of claim 1.

5. A method of inducing blood clotting comprising administering an effective amount of a pharmaceutical composition of claim 4 to a subject in need thereof.
